(19) United States
(12) Patent Application Publication
(10) Pub. No.: US 2019/0083578 A1
(43) Pub. Date: Mar. 21, 2019

(54) USE OF LOW DOSE GLUCAGON

(71) Applicant: XERIS PHARMACEUTICALS, INC., Austin, TX (US)

(72) Inventors: Yashvinder SABHARWAL, Austin, TX (US); Steven PRESTRELSKI, Austin, TX (US); Michael SANDOVAL, Austin, TX (US); Martin DONOVAN, Austin, TX (US)

(21) Appl. No.: 15/735,052

(22) PCT Filed: Jun. 10, 2016
(86) PCT No.: PCT/US2016/036921

§ 371 Date: Dec. 8, 2017

Related U.S. Application Data
(63) Continuation-in-part of application No. 15/136,650, filed on Apr. 22, 2016, now Pat. No. 9,649,364.
(60) Provisional application No. 62/233,032, filed on Sep. 25, 2015.
(60) Provisional application No. 62/173,457, filed on Jun. 10, 2015.

(51) Int. Cl. A61K 38/26; A61K 47/26; A61K 47/02; A61K 47/20; A61K 47/10; A61P 3/04

(52) U.S. Cl. CPC A61K 38/26; A61K 47/26; A61K 47/02; A61K 9/0024; A61K 47/10; A61P 3/04; A61K 47/20

(57) ABSTRACT

The present invention provides methods for controlling or reducing caloric intake in a subject by administering a low dose of a stable glucagon formulation. The present invention further provides methods for treating mild or moderate hypoglycemia in a subject in need thereof subject by administering a low dose of a stable glucagon formulation. Kits for practicing the methods of the invention are also provided.

### CROSS-REFERENCE WITH RELATED APPLICATIONS

This Application claims priority to U.S. Provisional patent application Ser. Nos. 62/173,457 filed Jun. 10, 2015 and 62/233,032 filed Sep. 25, 2015; and U.S. patent application Ser. No. 15/136,650 filed Apr. 22, 2016, each of which is incorporated herein by references in its entirety.

### FIELD OF THE INVENTION

The present invention relates to the fields of medicine, health, and nutrition. More particularly, the present invention relates to methods and compositions for treating diabetes and reducing or controlling body weight or altering body composition in a subject.

### BACKGROUND OF THE INVENTION

Diabetes is a serious metabolic disease in which the pancreas produces little or no insulin or the insulin that is present is not used effectively. As a result, blood glucose levels are chronically elevated in the diabetic individual. There are several main types of diabetes, including insulin-dependent (type 1) diabetes and non-insulin-dependent (type 2) diabetes. In type 1 diabetes, the pancreas does not produce sufficient levels of insulin for maintaining blood glucose at normal physiological levels. As a result, an individual having type 1 diabetes must take insulin daily. In type 2 diabetes, initially the pancreas generally produces enough insulin, but the body is unable to utilize the insulin effectively, a condition known as “insulin resistance.” However, as the disease progresses, insulin production decreases. Thus, individuals with an advanced stage of type 2 diabetes are often prescribed insulin in order to maintain blood glucose levels and minimize complications from the disease.

Chronic insulin therapy is often associated with weight gain. Furthermore, weight gain can be exacerbated by the ingestion of glucose in response to mild or moderate hypoglycemia. As a result, there is a need for methods of controlling or reducing body weight in diabetics. Moreover, obesity and weight gain is a serious health issue even among normoglycemic individuals, and therefore, there is also a need for methods of controlling or reducing body weight among individuals having normal blood glucose levels.

Glucagon is a peptide hormone that is secreted by the pancreas and which raises blood glucose levels. Glucagon has the opposite effect as insulin on blood glucose levels, and both glucagon and insulin are part of a feedback system that keeps blood glucose levels stable in the body. Glucagon is currently prescribed as a treatment for severe hypoglycemia, and smaller doses of glucagon are prescribed for treating mild to moderate hypoglycemia in cases of gastroparesis or other situations where ingestion of oral carbohydrates is not possible. Because glucagon is typically given by intramuscular, intravenous, or subcutaneous injection, it is necessary that the glucagon be in an injectable form. Currently available glucagon products must be reconstituted prior to use, a step that requires a sterile diluent to be injected into a vial containing powdered glucagon, because the hormone is highly unstable when dissolved in solution. When dissolved in a fluid state, glucagon can form amyloid fibrils, or tightly woven chains of proteins made up of individual glucagon peptides. Once glucagon begins to fibrillate, it becomes useless when injected, as the glucagon cannot be absorbed and used by the body.

As such, what is needed in the art are methods for providing a low dose of glucagon to a subject in need thereof, wherein the glucagon remains stable for an extended period of time once it is reconstituted. The present invention addresses this need and others.

### BRIEF SUMMARY OF THE INVENTION

In another aspect of the present invention there is disclosed a method for controlling or reducing body weight in a subject in need thereof. In certain aspects controlling or reducing body weight is by controlling or reducing caloric intake in a subject. In some embodiments, the method comprises: administering to the subject a low dose of a stable glucagon formulation in response to a hunger cue in the subject, wherein the glucagon formulation is stable for at least one week at controlled room temperature. In certain aspects, the methods act via controlling or reducing caloric intake in the subject. In some embodiments, the method comprises temporal or chronic administration of the glucagon formulation.

In some embodiments, the subject in need of controlling or reducing body weight is normoglycemic. In some embodiments, the subject is diabetic. In some embodiments, the subject is a human adult. In some embodiments, wherein the subject is a human adult, the glucagon formulation is administered to the subject at a dose of about 50 μg to about 200 μg. In some embodiments, the subject is a human child. In some embodiments, wherein the subject is a human child, the glucagon formulation is administered to the subject at a dose of about 5 μg to about 150 μg.

In another aspect, the present invention provides methods for treating mild or moderate hypoglycemia in a subject in need thereof. In some embodiments, the method comprises: administering to the subject a first low dose of a stable glucagon formulation in response to a symptom of mild or moderate hypoglycemia in the subject, wherein the glucagon formulation is stable for at least one week at controlled room temperature; thereby treating the mild or moderate hypoglycemia in the subject. In some embodiments, the method comprises chronic administration of the glucagon formulation.

In some embodiments, the subject in need of treating mild or moderate hypoglycemia is diabetic. In some embodiments, the subject is a human adult. In some embodiments, wherein the subject is a human adult, the glucagon formulation is administered to the subject at a dose of about 50 μg to about 200 μg. In some embodiments, the subject is a human child. In some embodiments, wherein the subject is a human child, the glucagon formulation is administered to the subject at a dose of about 5 μg to about 150 μg.

In some embodiments, the method further comprises: monitoring the subject for one or more symptoms of mild or moderate hypoglycemia subsequent to administering the first low dose of the glucagon formulation; and, if about 30 minutes after administering the first low dose of the glucagon formulation the subject exhibits one or more symptoms of mild or moderate hypoglycemia, administering to the subject a second low dose of the glucagon formulation. The steps of monitoring the subject for one or more symptoms of mild or moderate hypoglycemia and administering a subsequent low dose of glucagon can be repeated two, three, four, five, or more times over a defined period of time in order to treat the mild or moderate hypoglycemia in the subject.

In some embodiments, the symptom of mild or moderate hypoglycemia is a blood glucose concentration of less than about 70 mg/dL. In some embodiments, the method further comprises: monitoring the blood glucose level of the subject subsequent to administering the first low dose of the glucagon formulation; and, if about 30 minutes after administering the first low dose of the glucagon formulation the blood glucose level of the subject is less than about 70 mg/dL, administering to the subject a second low dose of the glucagon formulation. The steps of monitoring the blood glucose concentration of the subject and administering a subsequent low dose of glucagon can be repeated two, three, four, five, or more times over a defined period of time in order to treat the mild or moderate hypoglycemia in the subject.

In some embodiments, the glucagon formulation for administration according to any of the methods of the present invention is stable for at least one month at controlled room temperature. In some embodiments, the glucagon formulation is stable for at least one week at 40° C. In some embodiments, the glucagon formulation is stable for at least three months at 4° C.

In some embodiments, the glucagon formulation for administration according to any of the methods of the present invention is reconstituted with a pharmaceutically acceptable carrier. In some embodiments, the pharmaceutically acceptable carrier is an aqueous carrier. In some embodiments, the pharmaceutically acceptable carrier is a non-aqueous carrier.

In certain aspects, the glucagon, glucagon analogue, or a salt form of either thereof, can be fully solubilized in an aprotic polar solvent. Therapeutic molecules typically require an optimal or beneficial ionization profile in order to exhibit prolonged stability when solubilized in an aprotic polar solvent system. An optimal or beneficial ionization profile of a therapeutic molecule may be obtained by direct dissolution of the therapeutic agent in an aprotic polar solvent system containing a specified concentration of at least one ionization stabilizing excipient. Compositions for use with the present invention are stable formulations containing glucagon or glucagon analogue solubilized in an aprotic polar solvent system. In certain aspects the glucagon or glucagon analogue does not need to be previously dried from a buffered aqueous solution prior to reconstitution in the aprotic polar solvent system.

In certain aspects glucagon or glucagon analogue is directly dissolved (e.g. a powder as received from a commercial manufacturer or supplier) along with an effective amount of an ionization stabilizing excipient for establishing an appropriate ionization of the glucagon or glucagon analogue in the aprotic polar solvent system.

In certain aspects, the ability to circumvent the need for drying the peptide from a buffered aqueous solution, for example via lyophilization, prior to reconstitution in the aprotic polar solvent system is anticipated to save considerable time and cost throughout the various product development stages. It is well-known that the development of a drying method is an expensive and time-intensive processing step that often must be tailored to each therapeutic molecule. Further, during manufacturing the ability to scale-up the drying step is complicated by the use of equipment and/or instruments that differ considerably from those employed at the lab-scale, where the processing steps were initially studied and optimized. Accordingly, the ability to prepare a stable therapeutic peptide formulation via direct dissolution of the active ingredient in the aprotic polar solvent system, in the absence of such a drying step, will facilitate scale-up and manufacturing by eliminating a costly and time-consuming processing step. Further, during drying the therapeutic agent is exposed to multiple stresses than can degrade the molecule, and stabilizing excipients (e.g., disaccharides such as trehalose and sucrose) are often added to the formulation primarily to protect against degradation of the active agent during the drying process. By eliminating or minimizing the drying step the use of additional stabilizing excipients, particularly those that are often included to provide stability during the drying step, may be minimized, thereby allowing for the overall formulation to be simplified.

Stable solutions of glucagon or glucagon analogue solubilized in non-aqueous aprotic polar solvents (e.g. DMSO), can be prepared by adding a specific predetermined amount of a compound, or combination of compounds, that function as an ionization stabilizing excipient. The amount can be determined by titration studies using glucagon or glucagon analogue and the ionization stabilizing excipient. Without wishing to be bound by theory, it is believed that the ionization stabilizing excipient can act as a proton source (e.g., a molecule that can donate a proton to the therapeutic molecule) in the aprotic polar solvent system that may protonate the ionogenic groups on glucagon or glucagon analogue such that the glucagon or glucagon analogue possesses an ionization profile having an improved physical and chemical stability in the aprotic polar solvent system.

Certain embodiments are directed to a formulation of glucagon or glucagon analogue comprising a therapeutic agent at a concentration of at least, at most, or about 0.1, 1, 10, 50, or 100 mg/mL to 150, 200, 300, 400, or 500 mg/ml or up to the solubility limit of the glucagon or glucagon analogue in the aprotic polar solvent system comprising a concentration of at least one ionization stabilizing excipient that provides physical and chemical stability to the therapeutic agent. The formulation can comprise an ionization stabilizing excipient at a concentration of at least, at most, or about 0.01, 0.1, 0.5, 1, 10, or 50 mM to 10, 50, 75, 100, 500, 1000 mM, or up to the solubility limit of the ionization stabilizing excipient in the aprotic polar solvent system. In certain aspects the ionization stabilizing excipient concentration is between 0.1 mM to 100 mM. In certain embodiments the ionization stabilizing excipient may be a suitable mineral acid, such as hydrochloric acid. In further aspects the ionization stabilizing excipient may be an organic acid, such as an amino acid, amino acid derivative, or the salt of an amino acid or amino acid derivative (examples include glycine, trimethylglycine (betaine), glycine hydrochloride, and trimethylglycine (betaine) hydrochloride). In a further aspect the amino acid can be glycine or the amino acid derivative trimethylglycine. In further aspects the aprotic solvent system comprises DMSO. The aprotic solvent can be deoxygenated, e.g., deoxygenated DMSO. In certain aspects the therapeutic agent is glucagon or salt thereof.

Compositions to be used in conjunction with the present invention can be made by: (a) calculating or determining the appropriate ionization stabilizing excipient or proton concentration needed to achieve a stabilizing ionization profile of glucagon or glucagon analogue in an aprotic polar solvent system; (b) mixing at least one ionization stabilizing excipient with the aprotic polar solvent system to attain an appropriate ionization environment that provides the ionization profile determined in step (a); and (c) solubilizing the glucagon or glucagon analogue in the aprotic solvent having an appropriate environment to physically and chemically stabilize the glucagon or glucagon analogue. In certain aspects the dissolution of the glucagon or glucagon analogue and the addition of the ionization stabilizing excipient to the aprotic polar solvent system can be done in any order or concurrently, thus the ionization stabilizing excipient can be mixed first followed by dissolution of the glucagon or glucagon analogue, or the glucagon or glucagon analogue can be dissolved followed by addition of the ionization stabilizing excipient to the solution, or the ionization stabilizing excipient and the glucagon or glucagon analogue can be added or dissolved in an aprotic polar solvent system concurrently. In a further aspect the entire amount of a component (e.g., glucagon or glucagon analogue or an ionization stabilizing excipient) need not to be mixed at a particular point; that is, a portion of the one or more components can be mixed first, second, or concurrently, and another portion mixed at another time, first, second, or concurrently. In certain aspects the ionization stabilizing excipient may be a suitable mineral acid, such as hydrochloric acid. The concentration of the glucagon or glucagon analogue and/or ionization stabilizing excipient added to the solution can be between 0.01, 0.1, 1, 10, 100, 1000 mM to its solubility limit, including all values and ranges there between. In certain aspects the aprotic polar solvent system is deoxygenated. In a further aspect the aprotic polar solvent system comprises, consists essentially of, or consists of DMSO or deoxygenated DMSO.

In some embodiments, the glucagon formulation for administration according to any of the methods of the present invention comprises: glucagon or a glucagon analog, or a salt thereof, that has been dried with a carbohydrate and a buffer having a pH of about 2.0 to about 3.5, wherein the glucagon is reconstituted with a pharmaceutically acceptable carrier. In some embodiments, the buffer is selected from a glycine buffer, a citrate buffer, a phosphate buffer, and mixtures thereof. In some embodiments, the carbohydrate is selected from trehalose, hydroxyethyl starch (HES), dextran, and mixtures thereof.

In some embodiments, the glucagon formulation for administration according to any of the methods of the present invention comprises: (a) glucagon or a glucagon analog, or a salt thereof, wherein the glucagon has been dried in a non-volatile buffer, and wherein the dried glucagon has a pH memory that is about equal to the pH of the glucagon in the non-volatile buffer, wherein the pH memory of the dried glucagon is from about 2.0 to about 3.0; and (b) an aprotic polar solvent; wherein the moisture content of the formulation is less than 5%, and wherein the dried glucagon maintains the pH memory that is about equal to the pH of the glucagon in the non-volatile buffer when the dried glucagon is reconstituted in the aprotic polar solvent. In some embodiments, the non-volatile buffer is selected from a glycine buffer, a citrate buffer, a phosphate buffer, and mixtures thereof. In some embodiments, the aprotic polar solvent is selected from dimethylsulfoxide (DMSO), n-methyl pyrrolidone (NMP), ethyl acetate, and mixtures thereof.

In some embodiments, the glucagon formulation for administration according to any of the methods of the present invention is administered subcutaneously. In some embodiments, the glucagon formulation is administered via the use of a syringe. In some embodiments, the glucagon formulation is administered via the use of a pen injection device. In some embodiments, the glucagon formulation is administered via the use of an auto-injector device. In some embodiments, the glucagon formulation is administered via the use of a needle-free injection device. In some embodiments, the glucagon formulation is administered via the use of a multi-dose injection device.

In yet another aspect, the present invention provides kits for administering a low dose of glucagon to a subject in need of controlling or reducing body weight by controlling or reducing caloric intake. In still another aspect, the present invention provides kits for administering a low dose of glucagon to a subject in need of treating mild or moderate hypoglycemia. In some embodiments, the kit comprises: (a) a stable glucagon formulation, wherein the glucagon formulation is stable for at least one week at controlled room temperature; (b) a multi-dose cartridge or syringe; and (c) a multi-dose injection device capable of accepting the multi-dose cartridge or syringe.

In some embodiments, the kit comprises a glucagon formulation that is stable for at least one month at controlled room temperature. In some embodiments, the glucagon formulation is stable for at least one week at 40° C. In some embodiments, the glucagon formulation is stable for at least three months at 4° C.

In some embodiments, the kit comprises a glucagon formulation is reconstituted with a pharmaceutically acceptable carrier. In some embodiments, the pharmaceutically acceptable carrier is an aqueous carrier. In some embodiments, the pharmaceutically acceptable carrier is a non-aqueous carrier.

In some embodiments, the kit comprises a glucagon formulation comprising glucagon or a glucagon analogue at a concentration of at least, at most, or about 0.1, 1, 10, 50, or 100 mg/mL to 150, 200, 300, 400, or 500 mg/ml or up to the solubility limit of the glucagon or glucagon analogue in an aprotic polar solvent system comprising a concentration of at least one ionization stabilizing excipient that provides physical and chemical stability to the therapeutic agent. The formulation can comprise an ionization stabilizing excipient at a concentration of at least, at most, or about 0.01, 0.1, 0.5, 1, 10, or 50 mM to 10, 50, 75, 100, 500, 1000 mM, or up to the solubility limit of the ionization stabilizing excipient in the aprotic polar solvent system. In certain aspects the ionization stabilizing excipient concentration is between 0.1 mM to 100 mM. In certain embodiments the ionization stabilizing excipient may be a suitable mineral acid, such as hydrochloric acid. In further aspects the ionization stabilizing excipient may be an organic acid, such as an amino acid, amino acid derivative, or the salt of an amino acid or amino acid derivative (examples include glycine, trimethylglycine (betaine), glycine hydrochloride, and trimethylglycine (betaine) hydrochloride). In a further aspect the amino acid can be glycine or the amino acid derivative trimethylglycine. In further aspects the aprotic solvent system comprises DMSO. The aprotic solvent can be deoxygenated, e.g., deoxygenated DMSO. In certain aspects the therapeutic agent is glucagon or salt thereof.

In some embodiments, the kit comprises a glucagon formulation comprising: glucagon or a glucagon analog, or a salt thereof, that has been dried with a carbohydrate and a buffer having a pH of about 2.0 to about 3.5, wherein the glucagon is reconstituted with a pharmaceutically acceptable carrier. In some embodiments, the buffer is selected from a glycine buffer, a citrate buffer, a phosphate buffer, and mixtures thereof. In some embodiments, the carbohydrate is selected from trehalose, hydroxyethyl starch (HES), dextran, and mixtures thereof.

In some embodiments, the kit comprises a glucagon formulation comprising: (a) glucagon or a glucagon analog, or a salt thereof, wherein the glucagon has been dried in a non-volatile buffer, and wherein the dried glucagon has a pH memory that is about equal to the pH of the glucagon in the non-volatile buffer, wherein the pH memory of the dried glucagon is from about 2.0 to about 3.0; and (b) an aprotic polar solvent; wherein the moisture content of the formulation is less than 5%, and wherein the dried glucagon maintains the pH memory that is about equal to the pH of the glucagon in the non-volatile buffer when the dried glucagon is reconstituted in the aprotic polar solvent. In some embodiments, the non-volatile buffer is selected from a glycine buffer, a citrate buffer, a phosphate buffer, and mixtures thereof. In some embodiments, the aprotic polar solvent is selected from dimethylsulfoxide (DMSO), n-methyl pyrrolidone (NMP), ethyl acetate, and mixtures thereof.

In some embodiments, the kit comprises glucagon that is formulated for dosing a human adult, wherein the dose of the glucagon formulation that is administered is from about 50 μg to about 150 μg, or from about 100 μg to about 200 μg, e.g., about 50, about 60, about 70, about 80, about 90, about 100, about 110, about 120, about 130, about 140, about 150, about 160, about 170, about 180, about 190, or about 200 μg. In some embodiments, the kit comprises glucagon that is formulated for dosing a human child, wherein the dose of the glucagon formulation that is administered is from about 5 μg to about 150 μg,

In some embodiments, the kit comprises a multi-dose cartridge or syringe that is pre-filled with the glucagon formulation.

In some embodiments, the multi-dose injection device of a kit of the present invention is a pen injection device. In some embodiments, the multi-dose injection device is an auto-injector device. In some embodiments, the multi-dose injection device is a pump, e.g., a glucagon infusion pump or a patch pump. In some embodiments, the multi-dose injection device is an implantable injection device. In some embodiments, the multi-dose injection device is a needle-free injection device. In some embodiments, the multi-dose injection device is a variable dose device. In some embodiments, the multi-dose injection device is a low volume injection device.

In some embodiments, the kit further comprises instructions for administering the glucagon formulation. In some embodiments, the kit comprises instructions for using the glucagon formulation for treating hypoglycemia. In some embodiments, the kit comprises instructions for using the glucagon formulation for controlling or reducing weight or caloric intake.

### BRIEF DESCRIPTION OF THE DRAWINGS

FIG. 1. Size exclusion high-performance liquid chromatography performed on reconstituted commercially available glucagon samples after 4-6 hours incubation at refrigerated temperatures.

FIG. 2. Reverse phase high-performance liquid chromatography performed on reconstituted commercially available glucagon samples after 4-6 hours incubation at refrigerated temperatures.

FIG. 3. Observed Weigh-Gain During Dosing with either vehicle, or glucagon formulation at 0.5, 1.0, or 2.0 mg/kg/day.

FIG. 4. Food Consumption During Treatment with Glucagon

### DETAILED DESCRIPTION OF THE INVENTION

In one aspect, the present invention relates to the use of low dose glucagon therapy as a method of treating mild or moderate hypoglycemia in a subject. Currently commercially available glucagon formulations for use in low dose glucagon therapy must be reconstituted immediately prior to use, and are only stable for a few hours post-reconstitution before the glucagon fibrillates. Thus, it is impractical to reconstitute glucagon for the chronic use of glucagon in treating mild or moderate hypoglycemia. Instead, mild or moderate hypoglycemia is routinely treated by eating or drinking oral carbohydrates. However, chronic consumption of glucose-rich food, over time, can result in weight gain, which in turn can result in or contribute to other serious health conditions.

The stable glucagon formulations described herein have the advantage of significantly increased stability over time at ambient or even physiological temperatures. Because the stable glucagon formulations described herein have a significantly longer shelf life than currently available reconstituted glucagon formulations, these stable glucagon formulations can be used for the chronic administration of low dose glucagon therapy in place of ingestion of oral carbohydrates. By administering a stable glucagon formulation as described herein instead of high calorie, glucose-rich food during episodes of mild or moderate hypoglycemia, caloric intake and body weight can be controlled or reduced. Thus, the present invention also relates to the use of low dose glucagon therapy as a method of controlling or reducing caloric intake and/or body weight. This method of replacing a higher-calorie food or drink with a low dose of glucagon, thereby controlling or reducing caloric intake and/or body weight, is useful not only for individuals in need of controlling blood glucose levels (e.g., individuals having a diabetic condition), but for any individual who wants to control or reduce caloric intake.

### I. Definitions

For the purposes of the present disclosure, the following terms have the following meanings:

The term “low dose,” as used with respect to a dose of a stable glucagon formulation as described herein, means a dose of about 0.1 μg/kg to about 5 μg/kg. In some embodiments, a low dose of glucagon is a dose that is from about 5 μg to about 200 μg. One of skill in the art will recognize that a low dose of glucagon that is administered to an adult will be proportionately smaller for administration to a child, based on the age and/or size of the child.

The term “stable formulation” means that at least about 70% chemically and physically stable therapeutic agent (e.g., glucagon) remains after two months of storage at room temperature. Particularly preferred formulations are those in which at least about 80%, 85%, 90%, 91%, 92%, 93%, 94%, 95%, 96%, 97%, 98%, or 99% chemically and physically stable therapeutic agent remains under these storage conditions. Especially preferred stable formulations are those which do not exhibit degradation after sterilizing irradiation (e.g., gamma, beta or electron beam).

The term “chemical stability” means that with respect to a therapeutic agent (e.g., glucagon), an acceptable percentage of degradation products produced by chemical pathways such as oxidation or hydrolysis is formed. In particular, a formulation is considered chemically stable if no more than about 20% breakdown products are formed after one year of storage at the intended storage temperature of the product (e.g., room temperature); or storage of the product at 30° C./60% relative humidity for one year; or storage of the product at 40° C./75% relative humidity for one month, and preferably three months. In some embodiments, a chemically stable formulation has less than 20%, less than 15%, less than 10%, less than 5%, less than 4%, less than 3%, less than 2%, or less than 1% breakdown products formed after an extended period of storage at the intended storage temperature of the product.

The term “physical stability” means that with respect to a therapeutic agent (e.g., glucagon), an acceptable percentage of aggregates (e.g., dimers, trimers and larger forms that appear as amyloid fibrils) is formed. In particular, a formulation is considered physically stable if no more that about 15% aggregates are formed after one year of storage at the intended storage temperature of the product (e.g., room temperature); or storage of the product at 30° C./60% relative humidity for one year; or storage of the product at 40° C./75% relative humidity for one month, and preferably three months. In some embodiments, a physically stable formulation has less than 15%, less than 10%, less than 5%, less than 4%, less than 3%, less than 2%, or less than 1% aggregates formed after an extended period of storage at the intended storage temperature of the product.

The term “pharmaceutically acceptable carrier” refers to a pharmaceutically acceptable solvent, suspending agent, or vehicle for delivering a therapeutic agent (e.g., glucagon) to a human or animal. The carrier may be liquid, semisolid, or solid.

A “pharmaceutically acceptable” ingredient, excipient, or component is one that is suitable for use with humans and/or animals without undue adverse side effects (such as toxicity, irritation and allergic response) commensurate with a reasonable benefit/risk ratio.

The term “aprotic polar solvent” refers to a polar solvent that does not contain acidic hydrogen and does not act as a hydrogen bond donor. Examples of aprotic polar solvents include, but are not limited to, dimethylsulfoxide (DMSO), dimethylformamide (DMF), ethyl acetate, n-methyl pyrrolidone (NMP), dimethylacetamide (DMA), and propylene carbonate. The term aprotic polar solvent also encompasses mixtures of two or more aprotic polar solvents, e.g., a mixture of two or more of dimethylsulfoxide (DMSO), dimethylformamide (DMF), ethyl acetate, n-methyl pyrrolidone (NMP), dimethylacetamide (DMA), and propylene carbonate.

The term “controlled room temperature” refers to a temperature maintained thermostatically that encompasses the usual and customary working environment of 20° C. to 25° C. that allows for brief deviations between 15° C. and 30° C.

The term “administering” means oral (“po”) administration, administration as a suppository, topical contact, intravenous (“iv”), intraperitoneal (“ip”), intramuscular (“im”), intralesional, intranasal, or subcutaneous (“sc”) administration, or the implantation of a slow-release device e.g., a mini-osmotic pump, to a subject. Administration can be by any route including parenteral and transmucosal (e.g., oral, nasal, vaginal, rectal, or transdermal). Parenteral administration includes, e.g., intravenous, intramuscular, intraarterial, intradermal, subcutaneous, intraperitoneal, intraventricular, and intracranial administration. The term “intracutaneous” encompasses administration into the epidermal, dermal or subcutaneous skin layer.

The terms “treat” or “treatment” refer to delaying the onset of, retarding or reversing the progress of, or alleviating or preventing either the disease or condition to which the term applies, such as hypoglycemia, or one or more symptoms of such disease or condition.

The terms “controlling caloric intake” or “reducing caloric intake” refer to reducing the net caloric intake of an individual over a defined period of time, for example, reducing the net caloric intake of an individual on a daily basis, or over a period of days, weeks, or months., e.g., over 1, 2, 3, 4, 5, 6, 7, 8, 9, 10, or more weeks, or over 1, 2, 3, 4, 5, 6, 7, 8, 9, 10, or more months. In some embodiments, caloric intake is controlled or reduced in an individual treated with a stable glucagon formulation according to the methods of the present invention when the number of calories consumed per day by the individual is decreased by at least 1%, 2%, 3%, 4, %, 5%, 6%, 7%, 8%, 9%, 10%, 15%, 20%, 25%, 30%, 35%, 40%, 45%, 50%, or more as compared to the daily calorie consumption of the individual prior to treatment with the stable glucagon formulation. In some embodiments, caloric intake is controlled or reduced in an individual treated with a stable glucagon formulation according to the methods of the present invention when the net caloric intake of the individual is decreased by at least 1%, 2%, 3%, 4, %, 5%, 6%, 7%, 8%, 9%, 10%, 15%, 20%, 25%, 30%, 35%, 40%, 45%, 50% or more as compared to the caloric intake of the individual prior to treatment with the stable glucagon formulation.

The terms “patient,” “subject,” and “individual” interchangeably refer to a mammal, for example, a human or a non-human mammal, e.g., a primate, dog, cat, bovine, ovine, porcine, equine, mouse, rat, hamster, rabbit, or guinea pig.

The term “dissolution” as used herein refers to a process by which a material(s) in a gas, solid, or liquid state becomes a solute(s), a dissolved component(s), of a solvent, forming a solution of the gas, liquid, or solid in the solvent. In certain aspects a therapeutic agent, e.g., glucagon, or an excipient, e.g., an ionization stabilizing excipient, is present in an amount up to its solubility limited or is fully solubilized. The term “dissolve” refers to a gas, liquid, or solid becoming incorporated into a solvent to form a solution.

The term “excipient” as used herein refers to a natural or synthetic substance formulated alongside the active or therapeutic ingredient (an ingredient that is not the active ingredient) of a medication, included for the purpose of stabilization, bulking, or to confer a therapeutic enhancement on the active ingredient in the final dosage form, such as facilitating drug absorption, reducing viscosity, enhancing solubility, adjusting tonicity, mitigating injection site discomfort, depressing the freezing point, or enhancing stability. Excipients can also be useful in the manufacturing process, to aid in the handling of the active substance concerned such as by facilitating powder flowability or non-stick properties, in addition to aiding in vitro stability such as prevention of denaturation or aggregation over the expected shelf life.

As used herein an “ionization stabilizing excipient” is an excipient that establishes and/or maintains a particular ionization state for a therapeutic agent. In certain aspects the ionization stabilizing excipient can be, or includes, a molecule that donates at least one proton under appropriate conditions or is a proton source. According to the Bronsted-Lowry definition, an acid is a molecule that can donate a proton to another molecule, which by accepting the donated proton may thus be classified as a base. As used in this application, and as will be understood by the skilled technician, the term “proton” refers to the hydrogen ion, hydrogen cation, or H⁺. The hydrogen ion has no electrons and is composed of a nucleus that typically consists solely of a proton (for the most common hydrogen isotope, protium). Specifically, a molecule that can donate a proton to the therapeutic agent is considered an acid or proton source, regardless of whether it is completely ionized, mostly ionized, partially ionized, mostly unionized, or completely unionized in the aprotic polar solvent.

As used herein a “mineral acid” is an acid that is derived from one or more inorganic compounds. Accordingly, mineral acids may also be referred to as “inorganic acids.” Mineral acids may be monoprotic or polyprotic (e.g. diprotic, triprotic, etc.). Examples of mineral acids include hydrochloric acid (HCl), sulfuric acid (H₂SO₄), and phosphoric acid (H₃PO₄).

As used herein an “organic acid” is an organic compound with acidic properties (i.e. can function as a proton source). Carboxylic acids are one example of organic acids. Other known examples of organic acids include, but are not limited to, alcohols, thiols, enols, phenols, and sulfonic acids. Organic acids may be monoprotic or polyprotic (e.g. diprotic, triprotic, etc.).

“Charge profile,” “charge state,” “ionization state,” and “ionization profile” may be used interchangeably and refer to the ionization state (i.e. due to protonation and/or deprotonation) of the peptide's ionogenic groups.

The term “glucagon” refers to the glucagon peptide, analogues thereof, and salt forms of either thereof.

“Reconstituted,” when referring to a pharmaceutical composition, refers to a composition which has been formed by the addition of an appropriate non-aqueous solvent to a solid material comprising the active pharmaceutical ingredient. Pharmaceutical compositions for reconstitution are typically applied where a liquid composition with acceptable shelf-life cannot be produced. An example of a reconstituted pharmaceutical composition is the solution which results when adding a biocompatible aprotic polar solvent (e.g., DMSO) to a freeze dried composition.

“Analogue” and “analog,” when referring to a peptide, refers to a modified peptide wherein one or more amino acid residues of the peptide have been substituted by other amino acid residues, or wherein one or more amino acid residues have been deleted from the peptide, or wherein one or more amino acid residues have been added to the peptide, or any combination of such modifications. Such addition, deletion or substitution of amino acid residues can take place at any point, or multiple points, along the primary structure comprising the peptide, including at the N-terminal of the peptide and/or at the C-terminal of the peptide.

“Derivative,” in relation to a parent peptide, refers to a chemically modified parent peptide or an analogue thereof, wherein at least one substituent is not present in the parent peptide or an analogue thereof. One such non-limiting example is a parent peptide which has been covalently modified. Typical modifications are amides, carbohydrates, alkyl groups, acyl groups, esters, pegylations and the like.

“Mammal” or “mammalian” includes murine (e.g., rats, mice) mammals, rabbits, cats, dogs, pigs, and primates (e.g., monkey, apes, humans). In particular aspects in the context of the present invention, the mammal can be murine or human. The patient can be a mammal or a mammalian patient.

### II. Methods of Using Stable Glucagon Formulations

A. Methods for Controlling or Reducing Caloric Intake

In one aspect, the present invention provides methods for controlling or reducing caloric intake in a subject in need thereof. In some embodiments, the method comprises administering to the subject a low dose of a stable glucagon formulation in response to a hunger cue in the subject; thereby controlling or reducing caloric intake in the subject.

In some embodiments, the subject in need of controlling or reducing caloric intake is normoglycemic (i.e., has a normal concentration of glucose in the blood) or is non-diabetic. In some embodiments, the subject in need of controlling or reducing caloric intake is diabetic. As used herein, the term “diabetes” includes insulin-dependent type 1 diabetes, non-insulin-dependent type 2 diabetes, insulin-dependent type 2 diabetes, and gestational diabetes. In some embodiments, the subject has type 1 diabetes. In some embodiments, the subject has type 2 diabetes.

In some embodiments, the subject is a human adult. In some embodiments, the subject is a human child (i.e., an individual under the age of 18 years).

As used herein, a “hunger cue” refers to any physiological sensation or symptom that is manifested by the body when a subject is motivated to consume food. A hunger cue may include, but is not limited to, hunger pangs, lightheadedness or loss of balance, nausea, headaches, muscle weakness, lack of energy, or inability to concentrate.

Control of or reduction in caloric intake can be measured by any known method. In some embodiments, control of or reduction in caloric intake is determined by measuring the number of calories consumed in a day (e.g., in a 24-hour period). In some embodiments, caloric intake is controlled or reduced in an individual treated with a stable glucagon formulation according to the methods of the present invention when the number of calories consumed per day by the individual is decreased by at least 1%, 2%, 3%, 4, %, 5%, 6%, 7%, 8%, 9%, 10%, 15%, 20%, 25%, 30%, 35%, 40%, 45%, 50%, or more as compared to the daily calorie consumption of the individual prior to treatment with the stable glucagon formulation. The daily caloric intake can be measured over a defined period of time, e.g., over 1, 2, 3, 4, 5, 6, 7, 8, 9, 10, or more days; over 1, 2, 3, 4, 5, 6, 7, 8, 9, 10, or more weeks; or over 1, 2, 3, 4, 5, 6, 7, 8, 9, 10, or more months.

In other embodiments, control of or reduction in caloric intake is determined by measuring a reduction in body mass, e.g., body-mass index (BMI). The reduction in caloric intake (e.g., as measured by measuring a reduction in body mass) can be measured over a defined period of time. For example, in some embodiments, the reduction in caloric intake in an individual can be measured over 1, 2, 3, 4, 5, 6, 7, 8, 9, 10, or more weeks, or over 1, 2, 3, 4, 5, 6, 7, 8, 9, 10, or more months. In some embodiments, caloric intake is controlled or reduced in an individual treated with a stable glucagon formulation according to the methods of the present invention when the net caloric intake of the individual is decreased by at least 1%, 2%, 3%, 4, %, 5%, 6%, 7%, 8%, 9%, 10%, 15%, 20%, 25%, 30%, 35%, 40%, 45%, 50%, or more as compared to the caloric intake of the individual prior to treatment with the stable glucagon formulation. In some embodiments, caloric intake is reduced in an individual treated with a stable glucagon formulation according to the methods of the present invention when the BMI of the individual is reduced by at least 1%, 2%, 3%, 4%, 5%, 6%, 7%, 8%, 9%, 10%, 15%, 20%, 25%, or more as compared to the BMI of the individual prior to treatment with the stable glucagon formulation. In some embodiments, caloric intake is reduced in an individual treated with a stable glucagon formulation according to the methods of the present invention when the individual's body weight is decreased by at least about 5 lb, 10 lb, 15 lb, 20 lb, 25 lb or more as compared to the body weight of the individual prior to treatment with the stable glucagon formulation.

B. Methods for Treating Hypoglycemia

In another aspect, the present invention provides methods for treating mild or moderate hypoglycemia in a subject in need thereof. In some embodiments, the method comprises administering to the subject a low dose of a stable glucagon formulation in response to a symptom of mild or moderate hypoglycemia in the subject; thereby treating the mild or moderate hypoglycemia in the subject. In some embodiments, the method comprises chronic administration of a low dose of a stable glucagon formulation for treating mild or moderate hypoglycemia.

In some embodiments, the subject in need of treatment for mild or moderate hypoglycemia is diabetic. As used herein, the term “diabetes” includes insulin-dependent type 1 diabetes, insulin-dependent type 2 diabetes, non-insulin-dependent type 2 diabetes, and gestational diabetes. In some embodiments, the subject has type 1 diabetes. In some embodiments, the subject has type 2 diabetes.

In some embodiments, the subject is a human adult. In some embodiments, the subject is a human child (i.e., an individual under the age of 18 years).

In some embodiments, a subject is in need of treatment for mild or moderate hypoglycemia when the subject exhibits one or more symptoms of mild or moderate hypoglycemia. Symptoms of mild or moderate hypoglycemia include, but are not limited to, nausea; extreme hunger; cold, clammy, or wet skin or excessive sweating; rapid heartbeat; trembling; numbness or tingling of fingertips or toes; blurred vision; dizziness; headache; poor coordination; fatigue, lethargy, or drowsiness; and a blood glucose concentration of less than about 70 mg/dL. In some embodiments, the symptom of mild or moderate hypoglycemia is a blood glucose concentration of less than about 70 mg/dL. Blood glucose concentration can be measured according to any known method, including but not limited to, capillary blood glucose testing.

In some embodiments, the method of treating mild or moderate hypoglycemia further comprises, subsequent to administering a first low dose of the glucagon formulation, monitoring one or more symptoms of mild or hypoglycemia in the subject for a defined period of time (e.g., for about 15 minutes, about 30 minutes, about 45 minutes, about 60 minutes, or about 90 minutes); wherein if the subject exhibits one or more symptoms of mild or moderate hypoglycemia after the defined period of time, the method further comprises administering to the subject a second low dose of the glucagon formulation. The method may further comprise monitoring the subject for one or more symptoms of mild or moderate hypoglycemia subsequent to the second or subsequent dose of the glucagon formulation being administered, and wherein the subject continues to exhibit one or more symptoms of mild or moderate hypoglycemia after a defined period of time, administering a further low dose of the glucagon formulation. In some embodiments, the method comprises administering two, three, four, five, or more low doses of a glucagon formulation over a defined period of time (e.g., over the course of several hours) in order to treat the mild or moderate hypoglycemia in the subject.

In some embodiments, the method comprises monitoring one or more symptoms of mild or hypoglycemia in the subject by measuring the blood glucose level of the subject subsequent to administering a low dose of the glucagon formulation for a defined period of time, e.g., for about 15 minutes, about 30 minutes, about 45 minutes, about 60 minutes, or about 90 minutes. In some embodiments, the method comprises monitoring the blood glucose level of the subject for about 30 minutes; and, if about 30 minutes after administering the low dose of the glucagon formulation the blood glucose level of the subject is less than about 70 mg/dL, administering to the subject a further low dose of the glucagon formulation.

C. Methods of Administration

The stable glucagon formulation for administration according to the methods of the present invention can be any glucagon formulation that is stable for extended periods of time at controlled room temperature. In some embodiments, the glucagon formulation is stable for at least one week at controlled room temperature. In some embodiments, the glucagon formulation is stable for at least 2, 3, 4, 5, 6, 7, 8, 9, or 10 weeks or longer at controlled room temperature. In some embodiments, the stable glucagon formulation is stable for at least 1 month, or at least 2, 3, 4, 5, 6, 7, 8, 9, 10, 11, or 12 months or longer at controlled room temperature. In some embodiments, the stable glucagon formulation is a formulation that is functional over a range of temperatures. Suitable glucagon formulations for administration according to the methods of the present invention are described infra.

The amount of the glucagon formulation that is administered will vary according to several factors, including the formulation of the composition, patient response, the severity of the condition, the subject's weight, and the judgment of the prescribing physician. The dosage can be increased or decreased over time, as required by an individual patient. General guidance for appropriate dosages of all pharmacological agents used in the present methods is provided in Goodman and Gilman's The Pharmacological Basis of Therapeutics, 11th Edition, 2006, supra, and in a Physicians' Desk Reference (PDR), for example, in the 65th (2011) or 66th (2012) Eds., PDR Network, LLC, each of which is hereby incorporated herein by reference. In some embodiments, the dose of the glucagon formulation that is administered is from about 5 μg to about 200 μg, e.g., about 5, about 10, about 15, about 20, about 25, about 30, about 35, about 40, about 45, about 50, about 60, about 70, about 80, about 90, about 100, about 110, about 120, about 130, about 140, about 150, about 160, about 170, about 180, about 190, or about 200 μg.

In some embodiments, the subject is a human adult and the dose of the glucagon formulation that is administered is from about 50 μg to about 200 μg, e.g., about 50, about 60, about 70, about 80, about 90, about 100, about 110, about 120, about 130, about 140, about 150, about 160, about 170, about 180, about 190, or about 200 μg. In some embodiments, the subject is a human adult and the dose of the glucagon formulation that is administered is from about 50 μg to about 150 μg, or from about 100 μg to about 200 μg. In some embodiments, the subject is a human adult and the dose of the glucagon formulation that is administered is about 150 μg.

In some embodiments, the subject is a human child and the dose of the glucagon formulation that is administered is from about 5 μg to about 150 μg, from about 10 μg to about 150 μg, from about 15 μg to about 150 μg, or from about 20 μg to about 150 μg, e.g., about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, or about 150 μg. In some embodiments, the subject is a human child and the dose of the glucagon formulation that is administered is determined according to the following formula, wherein each “unit” of glucagon formulation is 10 μg: for a child ≤2 years of age, a dose of about 2 units (i.e., about 20 μg) is administered; for a child from 2-15 years of age, a dose of about one unit per each year of the child's age is administered (i.e., 2-15 units, i.e., from about 20 μg to about 150 μg); and for a child >15 years of age, a dose of about 15 units (i.e., about 150 μg) is administered.

In some embodiments, the glucagon formulation is administered chronically. As used herein, “chronic” administration refers to administration of a dose of a glucagon formulation one or more times per day for an extended period of time. In some embodiments, a glucagon formulation is chronically administered if a dose of a glucagon formulation is administered one or more times per day for at least 10 days, at least 20, 30, 40, 50, 60, 70, 80, 90, or 100 days, or for at least 150, 200, 250, 300, or 350 days, at least 1 year, or longer. In some embodiments, a glucagon formulation is chronically administered if at least 1, 2, 3, 4, 5, or more doses of a glucagon formulation are administered per day for at least 10 days, at least 20, 30, 40, 50, 60, 70, 80, 90, or 100 days or longer. In some embodiments, treatment can continue indefinitely. One of skill in the art will recognize that over the course of treatment, the number of doses of a glucagon formulation that is administered per day may vary.

Stable glucagon formulations can be administered by any method, including oral administration, administration as a suppository, transdermal, intravenous, intraperitoneal, intramuscular, intralesional, intranasal, or subcutaneous administration or by implantation. In some embodiments, a stable glucagon formulation for use in the present invention is administered by subcutaneous, intradermal, or intramuscular administration (e.g., by injection or by infusion). In some embodiments, the stable glucagon formulation is administered subcutaneously. In some embodiments, the stable glucagon formulation is administered by non-parenteral delivery, e.g., by nasal, buccal, or transdermal administration.

In some embodiments, a stable glucagon formulation is administered by infusion or by injection using any suitable device. For example, a glucagon formulation of the present invention may be administered via a syringe, a pen injection device, an auto-injector device, or a pump device (e.g., a glucagon infusion pump or a patch pump). In some embodiments, the injection device is an implantable injection device. In some embodiments, the injection device is a needle-free injection device. In some embodiments, the injection device is a multi-dose injector pump device or a multi-dose auto-injector device. In some embodiments, the multi-dose injection device (e.g., a multi-dose injector pump device or a multi-dose auto-injector device) is a variable dose device. In some embodiments, the injection device is a low volume injection device.

The formulation is presented in the device in such a fashion that the formulation is readily able to flow out of the needle upon actuation of an injection device, such as an auto-injector, in order to deliver the glucagon formulation. Suitable pen/autoinjector devices include, but are not limited to, those pen/autoinjection devices manufactured by Becton-Dickenson, Swedish Healthcare Limited (SHL Group), YpsoMed Ag, West Pharmaceuticals, Inc., and the like. Suitable pump devices include, but are not limited to, those pump devices manufactured by Tandem Diabetes Care, Inc., Delsys Pharmaceuticals, Insulet, Inc., Medtronics, Inc., and the like. Suitable needle-free injection devices include, but are not limited to, those devices manufactured by Zogenix, Inc., Bioject Medical Technologies, Inc., Antares Pharma, Inc., and the like.

A glucagon formulation for administration by infusion or injection can be formulated into a preparation suitable for injection or infusion by dissolving, suspending, or emulsifying the glucagon in a pharmaceutically acceptable carrier. The pharmaceutically acceptable carrier can be an aqueous carrier or a non-aqueous carrier. In some embodiments, the pharmaceutically acceptable carrier is a non-aqueous carrier including, but are not limited to, lipids, aryl benzonates, alkyl benzonates and triacetin. In some embodiments, the non-aqueous carrier is triacetin, benzyl benzoate, miglyol, palm oil or mineral oil. In some embodiments, the pharmaceutically acceptable carrier is an aqueous carrier (e.g., water or an aqueous buffer). In some embodiments, the pharmaceutically acceptable carrier is an aprotic polar solvent, including but not limited to, dimethylsulfoxide (DMSO), dimethylformamide (DMF), ethyl acetate, n-methyl pyrrolidone (NMP), dimethylacetamide (DMA), or propylene carbonate. In some embodiments, the glucagon formulation that is formulated with a pharmaceutically acceptable carrier (e.g., an aqueous carrier or a non-aqueous carrier) further comprises one or more surfactants, e.g., Tween® 20.

In some embodiments, the glucagon formulation is formulated as a gel. Methods of formulating pharmacologically active agents as gels for administration by injection are described, for example, in WO 2011/075623, the contents of which are incorporated by reference herein in its entirety. In other embodiments, the glucagon formulation is formulated as a fast-dissolving solid matrix, for example, as described in WO 2011/031462, the contents of which are incorporated by reference herein in its entirety.

D. Combination Therapy

The glucagon formulations and related methods of the present invention may also be used in combination with the administration of other therapies such as weight loss/reduced caloric intake type therapies. Non-limiting examples of such weight loss/reduced caloric intake type therapies include exercise, administration of drugs, etc. Non-limiting examples of weight loss/reduced caloric intake drugs include bupropion, leptin, lorcaserin hydrochloride, naltrexone, orlistat, phentermine, topiramate, GLP-1 (glucagon-like peptide-1 (GLP-1), a GLP-1 agonist, exenatide, or analogs thereof), or any combination of said drugs.

In one aspect, the glucagon formulation can be administered before the additional weight loss/reduced caloric intake therapy, simultaneously with the additional therapy, or after the additional therapy. If the glucagon formulation and the additional weight loss/reduced caloric intake therapy are administered separate, then a person having ordinary skill in the art would ensure that a significant period of time did not expire between the time of each administration, such that the glucagon formulation and the additional therapy would be able to exert an advantageously combined effect on the subject. Non-limiting examples of such administration times includes administering the stable glucagon formulation within 24 hours, 12 hours, 6 hours, 5 hours, 4 hours, 3 hours, 2 hours, 1 hour, 45 minutes, 30 minutes, or 15 minutes of administration of the additional therapy. Alternatively, the additional therapy can be administered within 24 hours, 12, hours, 6 hours, 5 hours, 4 hours, 3 hours, 2 hours, 1 hour, 45 minutes, 30 minutes, or 15 minutes of administration of the stable glucagon formulation.

In some situations, however, it may be desirable to extend the time period for administration significantly, where several days (2, 3, 4, 5, 6 or 7) to several weeks (1, 2, 3, 4, 5, 6, 7 or 8) lapse between the respective administrations. Further, effective combination therapy may be achieved with a single composition or pharmacological formulation that includes both the glucagon formulation and an additional therapy (e.g., weight loss/reduced caloric intake agent or drug), or with two distinct compositions or formulations, administered at the same time, wherein one composition includes the glucagon formulation of this invention, and the other includes the second drug or agent.

### III. Stable Glucagon Formulations

The methods of the present invention comprise administering to a subject in need thereof a low dose of a stable glucagon formulation. Suitable stable glucagon formulations for use in the present invention may comprise any glucagon formulation that is stable for at least one week at controlled room temperature. In some embodiments, the glucagon formulation is stable for at least 2 weeks, at least 3 weeks, or at least 4 weeks or longer at controlled room temperature, or at least 1 month, at least 2 months, at least 3 months, at least 4 months, at least 5 months, at least 6 months, at least 7 months, at least 8 months, at least 9 months, at least 10 months, at least 11 months, or at least 12 months or longer at controlled room temperature. In some embodiments, the glucagon formulation is stable for an extended period of time over a range of temperatures. For example, in some embodiments, the formulation is stable for at least 1, 2, 3, or 4 weeks, or at least 1, 2, 3, 4, 5, 6, 7, 8, 9, 10, 11, or 12 months or longer at 40° C. In other embodiments, the glucagon formulation is stable for at least 1, 2, 3, or 4 weeks, or at least 1, 2, 3, 4, 5, 6, 7, 8, 9, 10, 11, or 12 months or longer at 4° C.

Exemplary stable glucagon formulations for use in the present invention include any of the glucagon formulations described in WO 2012/059762; WO 2012/059764; US 2011/0237510; US 2012/0071817; US 2012/0046225; or U.S. application Ser. No. 13/417,073 (“Stable Formulations for Parenteral Injection of Peptide Drugs”), filed Mar. 9, 2012, the contents of each of which is incorporated by reference herein in its entirety, and/or any of the glucagon formulations described below.

A stable glucagon formulation for use in the present invention may comprise glucagon or a glucagon analog or peptidomimetic, or a salt thereof (e.g., glucagon acetate). In some embodiments, the glucagon is present in the formulation in an amount ranging from about 0.5 mg/mL to about 100 mg/mL (e.g., about 0.5, 1, 2, 3, 4, 5, 6, 7, 8, 9, 10, 15, 20, 25, 30, 35, 40, 45, 50, 55, 60, 65, 70, 75, 80, 85, 90, 95, or 100 mg/mL). In some embodiments, the glucagon is present in the formulation in an amount ranging from about 0.5 mg/mL to about 60 mg/mL, from about 10 mg/mL to about 50 mg/mL, from about 20 mg/mL to about 50 mg/mL, from about 5 mg/mL to about 15 mg/mL, or from about 0.5 mg/mL to about 2 mg/mL.

A. Non-Aqueous Ionization Stabilized Compositions

In certain embodiments the glucagon composition is a non-aqueous ionization stabilized composition. To address the physical and/or chemical instability that many therapeutic molecules exhibit in water, formulations may be prepared wherein the therapeutic agent is dissolved in a biocompatible non-aqueous liquid, such as an aprotic polar solvent. The use of aprotic polar solvents to prepare non-aqueous therapeutic formulations to inhibit many common degradation pathways, particularly those involving water, can significantly improve the stability of the solubilized or dissolved therapeutic molecule(s). However, problems still remain with the compositions and methods disclosed in the prior art. In particular, direct dissolution of a therapeutic molecule in an aprotic polar solvent is not a suitable approach for preparing stable compositions of most therapeutic molecules. For example, when solubilized directly in DMSO at a concentration of 5 mg/mL the peptide hormone glucagon will form insoluble aggregates within one day of storage at room temperature. For a composition comprising only glucagon and DMSO, 5 mg/mL corresponds to approximately 0.45% (w/w) of the peptide compound, indicating that at even relatively low concentrations, direct dissolution in an aprotic polar solvent system is by itself incapable of preventing physical aggregation and/or gelation of a therapeutic molecule. Moreover, therapeutic molecules that may not form insoluble aggregates in an aprotic polar solvent system may nonetheless be prone to chemical degradation when solubilized directly in an aprotic polar solvent system.

The drying process is well known to impose several stresses on the therapeutic molecule, and additional excipients (e.g., lyoprotectants such as trehalose and sucrose, and/or surfactants such as polysorbate 80) must be included in the aqueous solution in sufficient amounts to protect the therapeutic molecule, thereby increasing the cost and complexity of the formulation. Further, the drying process (e.g., spray drying, freeze drying) must often be optimized for a given therapeutic molecule, both at the lab-scale during initial research and development where the process is initially developed, and then during the manufacturing-scale as the process is scaled-up and transferred to instruments and facilities capable of producing commercial-scale batches. Consequently, the combination of initially developing and optimizing a drying process for a given therapeutic molecule, coupled with the time and costs associated with both transferring the method and incorporating an additional step in the manufacturing process can be very expensive. Thus, there is a need for a method of providing the therapeutic molecule(s) with an appropriate ionization profile in an aprotic polar solvent system without the requirement of drying the molecule from a buffered aqueous solution where the pH of the aqueous solution is set to provide an appropriate ionization profile for the molecule.

The solution resides in dissolving an ionization stabilizing excipient(s) directly in the aprotic polar solvent, coupled with dissolution of the peptide molecule or small molecule directly in the aprotic polar solvent solution. Without wishing to be bound by theory, it is believed that by providing a sufficient quantity of ionization stabilizing excipient to achieve an appropriate or optimal ionization profile of the therapeutic molecule, electrostatic repulsion between therapeutic molecules possessing the same charge polarity (i.e. negatively or positively charged) may be sufficient in magnitude to prevent physical degradation (e.g., via short-range hydrophobic interaction between molecules that lead to aggregation). This is especially important for molecules that exhibit a tendency to aggregate in solution, particularly as the concentration of the molecule in solution is increased. Further, by controlling and optimizing the extent of the ionization (i.e., protonation or deprotonation) of the therapeutic agent, chemical degradation can be minimized, as, for example, an excess of protonation may promote chemical instability via degradative reactions such as oxidation (for example, oxidation of methionine residues) and fragmentation (for example, cleavage of the peptide backbone). Accordingly, for some therapeutic molecules there may be an optimal or beneficial ionization profile achieved via protonation such that physical and/or chemical degradation reactions are minimized. For a therapeutic peptide, the extent of protonation required for stability, and thus the amount of the ionization stabilizing excipient required in the solution, will depend on, among other things, the primary structure (i.e., amino acid sequence) and the peptide concentration in the solution.

Without wishing to be bound by theory, it is thought that in order to exhibit enhanced or optimal stability and solubility when formulated in an aprotic polar solvent system, a therapeutic molecule may require a specific ionization profile. The ionization profile is the charge state acquired via protonation and/or deprotonation of the therapeutic molecule's ionogenic groups. For example, protonation of the ionogenic amino acid residues (e.g. arginine, lysine) comprising a therapeutic peptide will confer an overall positive charge on the molecules in solution. The relatively long-range electrostatic repulsions between positively charged peptide molecules may inhibit the short-range hydrophobic interactions that can result in physical aggregation and/or gelation. Thus, in the absence of sufficient protonation (i.e., an optimal or beneficial ionization profile), therapeutic molecules dissolved in an aprotic polar solvent system may be physically unstable and lead to the formation of soluble and/or insoluble aggregates.

Accordingly, it may be necessary to include at least one excipient in a sufficient concentration to function as an ionization stabilizing agent that is capable of imparting the ionization profile for improved physical and/or chemical stability to the active agent in the aprotic polar solvent system. An appropriate concentration of the ionization stabilizing excipient(s) to be added to the solution depends on several factors including, but not limited to, the chemical structure of the ionization stabilizing excipient, the chemical structure of the active agent(s), the concentration of the active(s), the solvent system used, the presence of co-solvents, and the presence of additional excipients or formulation components and their respective concentrations.

In certain aspects a composition may be prepared by first adding the ionization stabilizing excipient to the aprotic polar solvent system, followed by addition of the therapeutic molecule. Alternatively, the therapeutic molecule may initially be solubilized in the aprotic polar solvent system followed by addition of the ionization stabilizing excipient. In a further aspect, the ionization stabilizing excipient and the therapeutic molecule may be solubilized simultaneously in the aprotic polar solvent system.

Each molecule that functions as an ionization stabilizing excipient will exhibit a certain tendency to donate protons to the therapeutic molecule(s) in a given solvent system; this tendency to donate protons may be referred to as the relative acidic strength of the molecule. For a fixed concentration of a proton-donating molecule, (and for simplicity it is assumed only monoprotic molecules in this example) molecules that have a greater acidic strength will protonate the therapeutic molecule to a greater extent than a weaker acid. Accordingly, the concentration of a given proton-donating molecule (ionization stabilizing excipient) required to achieve an appropriate or optimal ionization profile for the therapeutic molecules will be inversely proportional to its acidic strength. These and other non-limiting aspects of the present invention are discussed herein.

In certain aspects the aprotic polar solvent can be deoxygenated prior to preparation of the formulation. Many different techniques can be used in the context of the present invention to deoxygenate or remove oxygen from aprotic polar solvents (degasification or deoxygenation). For instance, it is contemplated that deoxygenation can, but is not limited to, remove oxygen that is dissolved in a liquid aprotic polar solvent either by the liquid alone, by the liquid and other solute molecules (e.g. micelles, cyclodextrins, etc.), or by other solute molecules alone. Non-limiting examples of deoxygenation techniques include placing the aprotic polar solvent under reduced pressure and/or heating the liquid to decrease the solubility of dissolved gas, fractional distillation, membrane degasification, substitution by inert gas, using a reducing agent, freeze-pump-thaw cycling, or long time storage in a container with air-locks.

Once treated or deoxygenated, the aprotic polar solvents may have less than 0.1 mM of dissolved oxygen, preferably less than 0.05 mM of dissolved oxygen. Methods known to those of skill in the art can be used to determine the amount of dissolved oxygen in any given aprotic polar solvent (e.g., a dissolved oxygen meter or probe device can be used such as the Dissolved Oxygen Probe commercially available by Vernier (Beaverton, Oreg., USA)).

B. Stable Glucagon Formulations Dried with a Carbohydrate and a Buffer

In some embodiments, a stable glucagon formulation for use in the present invention is a formulation that has been dried with a carbohydrate and a buffer having a pH of about 2.0 to about 3.5. Drying the glucagon with the buffer and the carbohydrate helps to protect against chemical degradation of the glucagon and helps to preserve the α-helix of the glucagon. Thus, the glucagon when reconstituted (e.g., with a pharmaceutically acceptable carrier) is stable for an extended period of time and at elevated temperatures.

In some embodiments, the glucagon formulation comprises: glucagon or a glucagon analog, or a salt thereof, that has been dried with a carbohydrate and a buffer having a pH of about 2.0 to about 3.5. In some embodiments, the glucagon is reconstituted with a pharmaceutically acceptable carrier.

Suitable carbohydrates for use in drying the glucagon include sugars and starches. In some embodiments, the carbohydrate is selected from the group consisting of trehalose, hydroxyethyl starch (HES), dextran and mixtures thereof. In some embodiments, the carbohydrate is trehalose. In some embodiments, the carbohydrate is hydroxyethyl starch (HES). In some embodiments, the carbohydrate is a mixture of trehalose and hydroxyethyl starch (HES).

Suitable buffers for drying the glucagon include a glycine buffer, a citrate buffer, a phosphate buffer and mixture thereof. In some embodiments, the buffer is a glycine buffer. In some embodiments, the buffer is a citrate buffer.

In some embodiments, the pharmaceutically acceptable carrier is an aqueous carrier. In some embodiments, the aqueous carrier is water. In some embodiments, the pharmaceutically acceptable carrier is a non-aqueous carrier. In some embodiments, the non-aqueous carrier is selected from the group consisting of lipids, aryl benzonates, alkyl benzonates and triacetin.

In some embodiments, the stable glucagon formulation further comprises a surfactant that protects the glucagon from physical damage. In some embodiments, the surfactant is polysorbate 20 (e.g., Tween® 20) or polysorbate 80.

In some embodiments, the stable glucagon formulation further comprises at least one non-aqueous protic solvent. Examples of non-aqueous protic solvents include, but are not limited to, polyethylene glycol (PEG), propylene glycol (PG), polyvinylpyrrolidone (PVP), methoxypropylene glycol (MPEG), glycerol, glycofurol, and mixtures thereof.

Optionally, the stable glucagon formulation further comprises one or more of an antioxidant, a chelator, and a preservative. Suitable antioxidants include, but are not limited to, ascorbic acid, cysteine, methionine, monothioglycerol, sodium thiosulphate, sulfites, BHT, BHA, ascorbyl palmitate, propyl gallate, and vitamin E. Suitable chelators include, but are not limited to, EDTA, tartaric acid and salts thereof, glycerin, and citric acid and salts thereof. Suitable preservatives include, but are not limited to, benzyl alcohols, methyl parabens and propyl parabens.

C. Stable Glucagon Formulations Dried from a Non-Volatile Buffer

In some embodiments, a stable glucagon formulation for use in the present invention is a formulation that has been prepared by first freeze-drying the glucagon in a non-volatile buffer to a dry peptide powder. The dried glucagon has a defined “pH memory” of the pH of the glucagon in the non-volatile buffer from which the glucagon was dried. Once dried, the resulting glucagon powder is dissolved in an aprotic polar solvent, thereby forming a stable formulation having a moisture content of the formulation is less than 5%. The dried glucagon maintains its defined pH memory when reconstituted in the aprotic polar solvent, i.e., the pH of the glucagon when reconstituted in the aprotic polar solvent is about equal to the pH of the glucagon in the non-volatile buffer from which it was dried. In some embodiments, the pH of the reconstituted glucagon is about equal to the pH of the glucagon in the non-volatile buffer from which it was dried when the glucagon is reconstituted in an aprotic polar solvent that is within one pH unit of the pH of the glucagon in the non-volatile buffer from which it was dried. Thus, for example, if the glucagon had a pH of 3.0 in the non-volatile buffer from which it was dried, a pH memory of from 2.0 to 4.0 for the reconstituted glucagon would be within one pH unit, and thus the pH memory of the reconstituted glucagon would be about equal to the pH of the glucagon in the non-volatile buffer. In some embodiments, the pH of the reconstituted glucagon is about equal to the pH of the glucagon in the non-volatile buffer from which it was dried when the glucagon is reconstituted in an aprotic polar solvent that is within half of a pH unit of the pH of the glucagon in the non-volatile buffer from which it was dried. This glucagon formulation is stable for extended periods of time, is ready for use without the need for reconstitution at the time of use, and is functional over a range of temperatures.

In some embodiments, the glucagon formulation comprises: (a) glucagon or a glucagon analog, or a salt thereof, wherein the glucagon has been dried in a non-volatile buffer, and wherein the dried glucagon has a pH memory that is about equal to the pH of the glucagon in the non-volatile buffer, wherein the pH memory of the dried glucagon is from about 2.0 to about 3.0; and (b) an aprotic polar solvent; wherein the moisture content of the formulation is less than 5%, and wherein the dried glucagon maintains the pH memory that is about equal to the pH of the glucagon in the non-volatile buffer when the dried glucagon is reconstituted in the aprotic polar solvent.

The concept of “pH memory,” as used with reference to the glucagon formulations as described herein, relates to the resulting charge profile (protonation state) after drying the glucagon from a buffered aqueous solution (e.g., from a non-volatile buffer). The protonation state, and thus the solubility and stability of the glucagon, in very low or zero moisture non-aqueous solvents are affected by the aqueous pH of the glucagon solution before drying and the drying conditions employed. When the glucagon is dried in a buffer species in which both the acidic and basic components are non-volatile, the pH memory of the dried glucagon will be about equal to the pH of the glucagon in the non-volatile buffer. See, e.g., Enzymatic Reactions in Organic Media, Koskinen, A. M. P., and Klibanov, A. M., eds., Springer (1996). Furthermore, the pH of the buffered aqueous solution (e.g., non-volatile buffer) in which the glucagon is dried can be optimized to yield a pH memory for the glucagon that results in optimal stability, maximum solubility, and minimal degradation when the dried glucagon is subsequently reconstituted in an aprotic polar solvent. Because aprotic polar solvents do not have exchangeable protons, when the dried glucagon is reconstituted into an aprotic polar solvent, the reconstituted formulation will maintain the solubility and stability characteristics of the optimal pH memory. The pH memory can be measured in several ways. For example, pH memory can be measured by reconstituting the dried glucagon into un-buffered water and measuring the pH of the reconstituted glucagon with a pH indicator such as pH paper or a calibrated pH electrode. Alternatively, pH memory can be determined for glucagon that has been reconstituted in an aprotic polar solvent (e.g., DMSO) by adding at least 20% water to the aprotic polar solvent (e.g., DMSO) and measuring the pH with a pH indicator. See, e.g., Baughman and Kreevoy, “Determination of Acidity in 80% Dimethyl Sulfoxide-20% Water,” Journal of Physical Chemistry, 78(4):421-23 (1974). Measurement of pH in an aprotic polar solvent-water solution may require a small correction (i.e., no more than 0.2 pH unit as per Baughman and Kreevoy, supra). In some embodiments, the pH memory of the dried glucagon is from about 2.0 to about 3.0 (e.g., about 2.0, about 2.5, or about 3.0).

Suitable non-volatile buffers for drying the glucagon include, for example, glycine buffers, citrate buffers, phosphate buffers, and mixtures thereof. In some embodiments, the non-volatile buffer is a glycine buffer or a citrate buffer. In some embodiments, the non-volatile buffer is a glycine buffer. In some embodiments, the non-volatile buffer is a mixture of glycine buffer and citrate buffer. In some embodiments, the non-volatile buffer is a mixture of citrate buffer and phosphate buffer.

Optionally, the stability of the injectable formulation is further enhanced by the inclusion of one or more stabilizing agents or stabilizing excipients in the formulation. In some embodiments, the stabilizing agent or stabilizing excipient is added prior to drying the glucagon. In other embodiments, the dried glucagon is reconstituted with the stabilizing agent or stabilizing excipient in the aprotic polar solvent. In some embodiments, the stabilizing excipient is selected from sugars, starches, sugar alcohols, and mixtures thereof. Examples of suitable sugars for stabilizing excipients include, but are not limited to, trehalose, glucose, sucrose, etc. Examples of suitable starches for stabilizing excipients include, but are not limited to, hydroxyethyl starch (HES). Examples of suitable sugar alcohols for stabilizing excipients include, but are not limited to, mannitol and sorbitol. In some embodiments, the stabilizing excipient is present in the formulation in an amount that is about 1%, about 5%, about 10%, about 15%, about 20%, about 25%, about 30%, about 35%, about 40%, about 45%, about 50%, about 55%, or about 60% (w/v).

Once the glucagon and non-volatile buffer (and optionally the stabilizing excipient) are dried to a powder, the dried glucagon powder is dissolved or reconstituted in an aprotic polar solvent. In some embodiments, the aprotic polar solvent is selected from dimethylsulfoxide (DMSO), dimethylformamide (DMF), ethyl acetate, n-methyl pyrrolidone (NMP), dimethylacetamide (DMA), propylene carbonate, and mixtures thereof. In some embodiments, the aprotic polar solvent is a mixture of two or more of dimethylsulfoxide (DMSO), dimethylformamide (DMF), ethyl acetate, n-methyl pyrrolidone (NMP), dimethylacetamide (DMA), and propylene carbonate. Dimethylsulfoxide (DMSO), ethyl acetate, and n-methyl pyrrolidone (NMP) are particularly preferred aprotic polar solvents, each of which is a biocompatible solvent. In some embodiments, the aprotic polar solvent is dimethylsulfoxide (DMSO). In other embodiments, the aprotic polar solvent is n-methyl pyrrolidone (NMP). In other embodiments, the aprotic polar solvent is a mixture of dimethylsulfoxide (DMSO) and n-methyl pyrrolidone (NMP). In still other embodiments, the aprotic polar solvent is a mixture of dimethylsulfoxide (DMSO) and ethyl acetate.

In some embodiments, the dried peptide powder is reconstituted in an aprotic polar solvent that is “neat,” i.e., that does not contain a co-solvent. In some embodiments, the dried peptide powder is reconstituted in a solution that comprises an aprotic polar solvent and that does not contain water as a co-solvent. In some embodiments, the glucagon powder is reconstituted in an aprotic polar solvent that further comprises at least one co-solvent that depresses the freezing point of the formulation, wherein the co-solvent is a polar protic solvent. In some embodiment, the co-solvent is selected from ethanol, propylene glycol (PG), glycerol, and mixtures thereof. In some embodiments, the co-solvent is ethanol or PG. In some embodiments, a co-solvent is present in the formulation in an amount ranging from about 10% (w/v) to about 50% (w/v), e.g., about 10%, about 15%, about 20%, about 25%, about 30%, about 35%, about 40%, about 45%, or about 50% (w/v).

Such glucagon formulations have very little residual moisture and, thus, the glucagon remains stable over extended periods of time. In some embodiments, the stable glucagon formulation has a moisture content that is less than 5%. In some embodiments, the moisture content is less than 4%, less than 3%, less than 2%, less than 1%, less than 0.5%, less than 0.4%, less than 0.3%, less than 0.25%, less than 0.2%, less than 0.15%, less than 0.1%, less than 0.075%, less than 0.05%, less than 0.025%, or less than 0.01%.

As a non-limiting example, a stable glucagon formulation suitable for use according to the methods of the present invention may comprise: glucagon or a salt thereof (e.g., glucagon acetate), wherein the glucagon has been dried in a glycine buffer, and wherein the dried glucagon has a pH memory that is about 2.0, about 2.5, or about 3.0; and an aprotic polar solvent selected from DMSO, ethyl acetate, NMP, and mixtures thereof; wherein the moisture content of the formulation is less than about 1%. Optionally, the glucagon formulation may further comprise one or more stabilizing excipients selected from sugars, starches, and mixtures thereof, and/or one or more co-solvents selected from ethanol, propylene glycol, glycerol, and mixtures thereof.

D. Partially Volatile Buffer

In one aspect of the present invention a composition is a stable formulation that includes: (a) a peptide or a salt thereof that has been previously dried from an aqueous composition comprising a partially volatile buffer, a volatile buffer, a strong acid, or a strong base, or any combination thereof, wherein the dried peptide or salt thereof has a first ionization profile that corresponds to the peptide's optimal stability and solubility; and an aprotic polar solvent, wherein the dried peptide or salt thereof is reconstituted into an aprotic polar solvent and has a second ionization profile in the aprotic polar solvent, wherein the first and second ionization profiles are substantially the same, such as within 1 pH unit of one another. For a more detailed description see PCT application PCT/US2015/014756, which is incorporated herein by reference in its entirety. One non-limiting method for measuring the ionization state of the dry peptide includes reconstituting the dried peptide into un-buffered water and measuring the pH of the reconstituted peptide with a pH indicator such as pH paper or a calibrated pH electrode. One non-limiting method for measuring the ionization state of the peptide that has been reconstituted in the aprotic polar solvent includes adding at least 20% water to the aprotic polar solvent and measuring the pH with a pH indicator.

The peptide or salt thereof can have a third ionization profile when the peptide is in the aqueous composition prior to the aforementioned drying step. The third ionization profile can be different from the first or second ionization profiles by at least 1 pH unit (e.g., the aqueous composition can be formulated such that the pH of the aqueous composition compensates for the loss of counter-ions or buffer components or both during drying of said aqueous composition). Alternatively, the third ionization profile can be substantially the same as the first or second ionization profiles, such as within 1 pH unit of one another. One non-limiting method for measuring the ionization state of the peptide in the aqueous composition prior to said drying step is to measure the pH of the aqueous solution with a pH indicator. In some particular aspects, the aqueous composition is formulated such that the third ionization profile shifts to the first ionization profile during drying of said aqueous composition.

The dried peptide can be partially or fully solubilized within the aprotic polar solvent. Full solubilization can be obtained by adding the dried peptide to the aprotic polar solvent up to the solubility limit of said peptide. For partial solubilization, suspensions and pastes can be formed such that a percentage of the peptide is solubilized in the aprotic polar solvent and a percentage is suspended or dispersed within said aprotic polar solvent. The aqueous composition can include a partially volatile buffer, non-limiting examples of which include sodium acetate or ammonium phosphate or any combination thereof. The aqueous composition can include a volatile buffer, non-limiting examples of which include ammonium acetate, ammonium formate, ammonium carbonate, ammonium bicarbonate, pyridine acetate, pyridine formate, or triethylammonium acetate, or any combination thereof. The aqueous composition can include a strong acid, a non-limiting example of which includes hydrochloric acid. The aqueous composition can include a strong base, non-limiting examples of which include sodium hydroxide, potassium hydroxide, lithium hydroxide, or calcium hydroxide, or any combination thereof.

In certain aspects, the aqueous composition does not include any buffer or does not include a non-volatile buffer. Alternatively, the aqueous composition can include a mixture of different buffers. In one non-liming aspect, the mixture can include a mixture of non-volatile buffers, a mixture of partially volatile buffers, a mixture of volatile buffers, a mixture of non-volatile and partially volatile buffers, a mixture of non-volatile and volatile buffers, a mixture of partially volatile and volatile buffers, or a mixture of non-volatile, partially volatile, and volatile buffers. The drying step can be performed by lyophilization, spray drying, desiccation, thin-film freezing, spray freeze drying, or any combination thereof. The moisture or water content of the formulation can be less than 15%, 10%, 5%, 1%, or less.

Non-limiting examples of aprotic polar solvents includes dimethylsulfoxide (DMSO), n-methyl pyrrolidone (NMP), ethyl acetate, dimethylformamide (DMF), propylene carbonate, or mixtures thereof. The formulation can further include a co-solvent that depresses the freezing point of the formulation (e.g., ethanol, propylene glycol, glycerol, and mixtures thereof). The formulation can further include a stabilizing excipient (e.g., a sugar, a starch, or mixtures thereof). In preferred aspects, the peptide in the formulation is glucagon or a salt thereof. In instances where the peptide is glucagon or a salt thereof, the first or second ionization profiles can correspond to the ionization profile of glucagon when solubilized in an aqueous solution having a pH range of about 2 to 3. The third ionization profile can correspond to the ionization profile of glucagon when solubilized in an aqueous solution having a pH range of about 2 to 3 or can correspond to the ionization profile of glucagon when solubilized in an aqueous solution having a pH range of greater than 3, or greater than 3 to 14, or greater than 3 to 10, or greater than 3, 4, 5, 6, 7, 8, 9, 10, 11, 12, 13, or 14 or any range therein. Alternatively, the third ionization profile can correspond to the ionization profile of glucagon when solubilized in an aqueous solution having a pH range of less than 2, or less than 2 to 0 or less than 2 to 1 or 1 or 0 or any range therein. Also, the first ionization profile can be maintained by reconstituting the dried peptide or salt thereof in an organic solvent system comprising an organic solvent and an organic phase buffer prior to reconstituting said dried peptide or salt thereof into polar aprotic solvent.

The dried peptide or salt thereof can be reconstituted into the polar aprotic solvent with mixing the organic solvent system with the polar aprotic solvent. The organic solvent system can be separated from the polar aprotic solvent via separation methods known in the art. The organic solvent system can be substantially anhydrous (e.g., less than 1 wt. %, less than 0.5 wt. %, or less than 0.1 wt. % water) or anhydrous.

E. Preparation of Glucagon Formulations

For making the stable glucagon formulations described herein for use in the present invention, in some embodiments, the glucagon is processed in order to decrease its particle size by any pharmaceutically acceptable manner known to those skilled in the art. Various methods of particle size manipulation and/or reduction can be utilized in order to prepare the glucagon. Such particle size reduction procedures include, but are not limited to, comminuting processes (cutting, chopping, crushing, grinding, milling, micronizing, nanosizing, freeze drying, spray-freeze-drying, trituration, and microfluidization).

Spray-drying includes the steps of atomization of a solution containing one or more solid (e.g., therapeutic agent) via a nozzle spinning disk, or other device, followed by evaporation of the solvent from the droplets. The nature of the powder that results is the function of several variables including the initial solute concentration, size distribution of droplets produced and the rate of solute removal. The particles produced may comprise aggregates of primary particles which consist of crystals and/or amorphous solids depending on the rate and conditions of solvent removal.

A spray-drying process for preparing ultra-fine powders of biological macromolecules such as proteins, oligopeptides, high molecular weight polysaccharides, and nucleic acids is described in, for example, U.S. Pat. No. 6,051,256. Freeze-drying procedures are well known in the art, and are described, for example, in U.S. Pat. Nos. 4,608,764 and 4,848,094. Spray-freeze-drying processes are described, e.g., in U.S. Pat. No. 5,208,998. Other spray-drying techniques are described, for example, in U.S. Pat. Nos. 6,253,463; 6,001,336; 5,260,306; and PCT International Publication Nos. WO 91/16882 and WO 96/09814.

Lyophilization techniques are well known to those skilled in the art. Lyophilization is a dehydration technique that takes place while a product is in a frozen state (ice sublimation under a vacuum) and under a vacuum (drying by gentle heating). These conditions stabilize the product, and minimize oxidation and other degradative processes. The conditions of freeze drying permit running the process at low temperatures, therefore, thermally labile products can be preserved. Steps in freeze drying include pretreatment, freezing, primary drying, and secondary drying. Pretreatment includes any method of treating the product prior to freezing. This may include concentrating the product, formulation revision (i.e., addition of components to increase stability and/or improve processing), decreasing a high vapor pressure solvent, or increasing the surface area. Methods of pretreatment include: freeze concentration, solution phase concentration, and formulating specifically to preserve product appearance or to provide lyoprotection for reactive products, and are described, e.g., in U.S. Pat. No. 6,199,297. “Standard” lyophilization conditions, are described, e.g., in U.S. Pat. No. 5,031,336, and in “Freeze Drying of Pharmaceuticals” (DeLuca, Patrick P., J. Vac. Sci. Technol., Vol. 14, No. 1, January/February 1977); and “The Lyophilization of Pharmaceuticals: A Literature Review” (Williams, N. A., and G. P. Polli, Journal of Parenteral Science and Technology, Vol. 38, No. 2, March/April 1984).

In some embodiments, the lyophilization cycle is partially performed above the glass transition temperature (Tg) of the therapeutic agent formulation to induce a collapse of the mass to form a dense cake containing residue moisture. In other embodiments, the lyophilization cycle is carried out below the glass transition temperature in order to avoid a collapse in order to achieve a complete drying of the particles.

F. Semi-Solid Paste Formulations

Another stable glucagon formulation may be prepared by formulating human glucagon for delivery as a pharmaceutically acceptable semi-solid paste suspension for administration via intracutaneous injection. The following can be used to prepare such a formulation: (1) lyophilized powder development followed by biocompatible carrier development, where in the first step the therapeutic agent (i.e. glucagon, or a glucagon analog or a salt thereof (e.g. glucagon acetate)) is prepared as a powder (either alone, or with excipients); and (2) in the second step the powder is mixed with a biocompatible non-aqueous diluent that does not solubilize the powder, such that a semi-solid suspension of the drug particles in the diluent is prepared. This semi-solid suspension may be described as a pharmaceutically acceptable paste for administration via intracutaneous injection, where the solid phase (i.e. powder) promotes thermostability and enables very high concentrations of the therapeutic to be achieved, while the liquid phase (i.e. diluent) serves as a carrier to minimize friction through a needle to allow the formulation to be syringeable.

In certain aspects, the semi-solid formulation can further include a carrier (e.g., one or more polymers) which imparts thixotropic properties to the formulation. The therapeutic agent can be homogeneously incorporated into the thixotropic pharmaceutically acceptable carrier, and said formulation is in the form of a paste or slurry.

In certain aspects, the therapeutic agent is homogeneously contained within a pharmaceutically acceptable diluent. The diluent functions as a carrier for the drug-containing powder and is preferably biocompatible and is a non-solvent to the powder such that the two phases (solid and liquid) are maintained in the formulation. The carrier in certain embodiments fills the spaces between particles in a way that makes them flow while also serving as a lubricant to minimize friction through a needle. In certain embodiments, the carrier is selected from the group consisting of alkyl benzoates, aryl benzoates, aralkyl benzoates, triacetin, dimethyl sulfoxide (DMSO), N-methyl-2-pyrrolidone (NMP), alkanes, cyclic alkanes, chlorinated alkanes, fluorinated alkanes, perfluorinated alkanes, polyethylene glycol, and mixtures thereof.

Optionally, the stability of the injectable formulation can be further enhanced by the inclusion of one or more stabilizing agents or stabilizing excipients in the formulation prior to drying the glucagon to a powder. In some embodiments, the stabilizing excipient is selected from sugars, starches, sugar alcohols, amino acids, and mixtures thereof. Examples of suitable sugars for stabilizing excipients include, but are not limited to, trehalose, glucose, sucrose, etc. Examples of suitable starches for stabilizing excipients include, but are not limited to, hydroxyethyl starch (HES). Examples of suitable sugar alcohols for stabilizing excipients include, but are not limited to, mannitol and sorbitol. An examples of an amino acid includes, but are not limited to, glycine. In some embodiments, the stabilizing excipient is present in the formulation in an amount that is about 1%, about 5%, about 10%, about 15%, about 20%, about 25%, about 30%, about 35%, about 40%, about 45%, about 50%, about 55%, or about 60% (w/v).

As a non-limiting example, a stable semi-solid suspension of glucagon for administration via intracutaneous injection and suitable for use according to the methods of the present invention may comprise: glucagon or a salt thereof (e.g., glucagon acetate), wherein the glucagon has been dried (e.g. lyophilized) in a glycine buffer, and wherein the dried glucagon has a pH memory that is about 2.0, about 2.5, or about 3.0. Optionally, the glucagon formulation may further comprise one or more stabilizing excipients selected from sugars, starches, and mixtures thereof. Following preparation of the powder, the powder is blended with a biocompatible non-aqueous carrier using, for example, a planetary mixer, and where the percent solids content (i.e. percent solids by weight) of the semi-solid suspension can vary between 1 to 99% dependent upon the physicochemical properties of the powder.

Current injectable formulations of glucagon are composed of 1 mg of glucagon in 49 mg of lactose at pH less than 3.0. The formulation envisioned by the present invention provides an efficacious dose of glucagon in a minimal volume using as little stabilizer as possible to stabilize the glucagon during the freeze-drying powder development process as well as during the shelf life, and may be achieved by freeze-drying the glucagon at as high a concentration as possible. An example of such a formulation would be 1 mg of glucagon in 3 microliters total volume, producing a highly concentrated formulation of approximately 333 mg/mL.

In the above example the glucagon-containing powder was prepared via lyophilization (freeze-drying), but the powder may be prepared by other methods well known to those skilled in the art, examples of which include spray drying and spray freeze drying, as well as additional particle engineering technologies such as thin film freezing.

An additional formulation for weight loss is a semi-solid co-formulation of glucagon (or a glucagon analog, or a salt thereof) and GLP-1 (glucagon-like peptide-1 (GLP-1), a GLP-1 agonist, exenatide, or analogs thereof), where both therapeutic agents are prepared into separate powder (each powder containing a single therapeutic agent) that are then mixed together and suspended in biocompatible diluent to produce a pharmaceutically acceptable semi-solid suspension that is administered to the patient via intracutaneous injection. The glucagon ((or a glucagon analog, or a salt thereof) can be dried to a powder in a buffering system, that may also include stabilizing excipients, such that it has a pH memory between 1 to 4 and preferably between 2 to 3. The GLP-1 (glucagon-like peptide-1, a GLP-1 agonist, exenatide, or analogs thereof) can be dried to a powder in a buffering system that may also include stabilizing excipients, such that it has a pH memory between 3 to 6 and preferably between 4 to 5. The buffering systems may be dried, for example, via lyophilization. The two powders can then be mixed together using, for example, an orbital blender, to prepare a uniform blend and suspended in a biocompatible non-aqueous carrier that is a non-solvent for both powders. The powders are blended with the selected biocompatible non-aqueous carrier using, for example, a planetary mixer, and where the percent solids content (i.e. percent solids by weight) of the semi-solid suspension can vary between 1-99% dependent upon the physicochemical properties of the powders. As such, only a single formulation is necessary to administer both GLP-1 and glucagon to a patient. The molar ratio of glucagon to GLP-1 may be range between 0.1:1 to 100:1, and preferably between 0.5:1 to 10:1.

In certain aspects, the injectable formulation is in controlled (slow) release form. In such embodiments, for example, the formulation may comprise a pharmaceutically acceptable polymer (e.g. PLGA, PLA) in an amount effective to slow the release of the therapeutic agent(s) from said formulation upon administration via injection into the epidermal, dermal or subcutaneous layer of an animal. Additionally, or alternatively, the therapeutic agent(s) may be incorporated into liposomes or conjugated to or incorporated with polysaccharides and/or other polymers to provide a controlled release of the therapeutic agent from said formulation upon administration via injection into the epidermal, dermal or subcutaneous layer of an animal.

### IV. Kits

In yet another aspect, the present invention provides kits for administering a stable glucagon formulation according to the methods of the present invention. In some embodiments, the present invention provides kits for controlling or reducing body weight by controlling or reducing caloric intake in a subject in need thereof. In some embodiments, the present invention provides kits for treating mild or moderate hypoglycemia in a subject in need thereof. In some embodiments, the kit comprises: (a) a stable glucagon formulation, wherein the glucagon formulation is stable for at least one week at controlled room temperature; (b) a multi-dose cartridge or syringe; and (c) a multi-dose injection device capable of accepting the multi-dose cartridge or syringe.

A suitable glucagon formulation for use in a kit of the present invention can be any glucagon formulation described herein. In some embodiments, the kit comprises a stable glucagon formulation, wherein the glucagon formulation is stable for at least one week at controlled room temperature. In some embodiments, the kit comprises a glucagon formulation that is stable for an extended period of time over a range of temperatures, e.g., a glucagon formulation that is stable for at least 1, 2, 3, or 4 weeks or at least 1, 2, 3, 4, 5, 6, 7, 8, 9, 10, 11, or 12 months at 40° C., or a glucagon formulation that is stable for at least 1, 2, 3, or 4 weeks or at least 1, 2, 3, 4, 5, 6, 7, 8, 9, 10, 11, or 12 months at 4° C.

In some embodiments, the kit comprises a stable glucagon formulation wherein the glucagon formulation comprises: glucagon or a glucagon analog, or a salt thereof, that has been dried with a carbohydrate and a buffer having a pH of about 2.0 to about 3.5, wherein the glucagon is reconstituted with a pharmaceutically acceptable carrier. In some embodiments, the glucagon formulation comprises glucagon that has been dried with trehalose and/or HES. In some embodiments, the glucagon is reconstituted with a non-aqueous carrier selected from the group consisting of lipids, aryl benzonates, alkyl benzonates, and triacetin. In some embodiments, the glucagon formulation further comprises a surfactant, including but not limited to Tween® 20.

In some embodiments, the kit comprises a stable glucagon formulation wherein the glucagon formulation comprises: (a) glucagon or a glucagon analog, or a salt thereof, wherein the glucagon has been dried in a non-volatile buffer, and wherein the dried glucagon has a pH memory that is about equal to the pH of the glucagon in the non-volatile buffer, wherein the pH memory of the dried glucagon is from about 2.0 to about 3.0; and (b) an aprotic polar solvent; wherein the moisture content of the formulation is less than 5%, and wherein the dried glucagon maintains the pH memory that is about equal to the pH of the glucagon in the non-volatile buffer when the dried glucagon is reconstituted in the aprotic polar solvent. In some embodiments, the non-volatile buffer is selected from a glycine buffer, a citrate buffer, a phosphate buffer, and mixtures thereof. In some embodiments, the aprotic polar solvent is selected from dimethylsulfoxide (DMSO), n-methyl pyrrolidone (NMP), ethyl acetate, and mixtures thereof. In some embodiments, the glucagon formulation further comprises a co-solvent that depresses the freezing point of the formulation, and/or a stabilizing excipient.

In some embodiments, the multi-dose cartridge or syringe is pre-filled with the glucagon formulation. In some embodiments, the multi-dose cartridge is pre-filled with 1, 2, 3, 4, 5, 6, 7, 8, 9, 10 or more doses of the glucagon formulation. Glucagon formulation doses can be any dose described herein, for example, a dose of from about 5 μg to about 200 μg, e.g., about 5, about 10, about 15, about 20, about 25, about 30, about 35, about 40, about 45, about 50, about 60, about 70, about 80, about 90, about 100, about 110, about 120, about 130, about 140, about 150, about 160, about 170, about 180, about 190, or about 200 μg. One of skill in the art will recognize that the glucagon formulation dose can be varied (e.g., increased or decreased over time), as required by an individual patient.

In some embodiments, the kit comprises glucagon that is formulated for dosing a human adult, wherein the dose of the glucagon formulation that is administered is from about 50 μg to about 150 μg, or from about 100 μg to about 200 μg. In some embodiments, the kit comprises glucagon that is formulated for dosing a human child, wherein the dose of the glucagon formulation that is administered is from about 5 μg to about 150 μg, from about 10 μg to about 150 μg, from about 15 μg to about 150 μg, or from about 20 μg to about 150 μg, e.g., about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, or about 150 μg.

In some embodiments, the multi-dose injection device capable of accepting the multi-dose cartridge or syringe is a pen injection device, an auto-injector device, or a pump (e.g., a glucagon infusion pump or a patch pump). In some embodiments, the multi-dose injection device is an implantable injection device. In some embodiments, the multi-dose injection device is a needle-free injection device. In some embodiments, the multi-dose device (e.g., a pen injection device, auto-injector device, or pump) is a variable dose device. In some embodiments, the multi-dose injection device is a low volume injection device.

Suitable pen/autoinjector devices for use in the kits of the present invention include, but are not limited to, those pen/autoinjection devices manufactured by Becton-Dickenson, Swedish Healthcare Limited (SHL Group), YpsoMed Ag, West Pharmaceuticals, Inc., and the like. Suitable pump devices include, but are not limited to, those pump devices manufactured by Tandem Diabetes Care, Inc., Delsys Pharmaceuticals, Insulet, Inc., Medtronics, Inc., and the like. Suitable needle-free injection devices include, but are not limited to, those devices manufactured by Zogenix, Inc., Bioject Medical Technologies, Inc., Antares Pharma, Inc., and the like.

In some embodiments, the kit further comprises instructions, wherein the instructions direct the administration of the glucagon formulation to control or reduce body weight in the subject in need thereof and/or to treat mild or moderate hypoglycemia in the subject in need thereof.

### Examples

The following examples are offered to illustrate, but not to limit, the claimed invention.

### Stability Assay for Glucagon Formulations

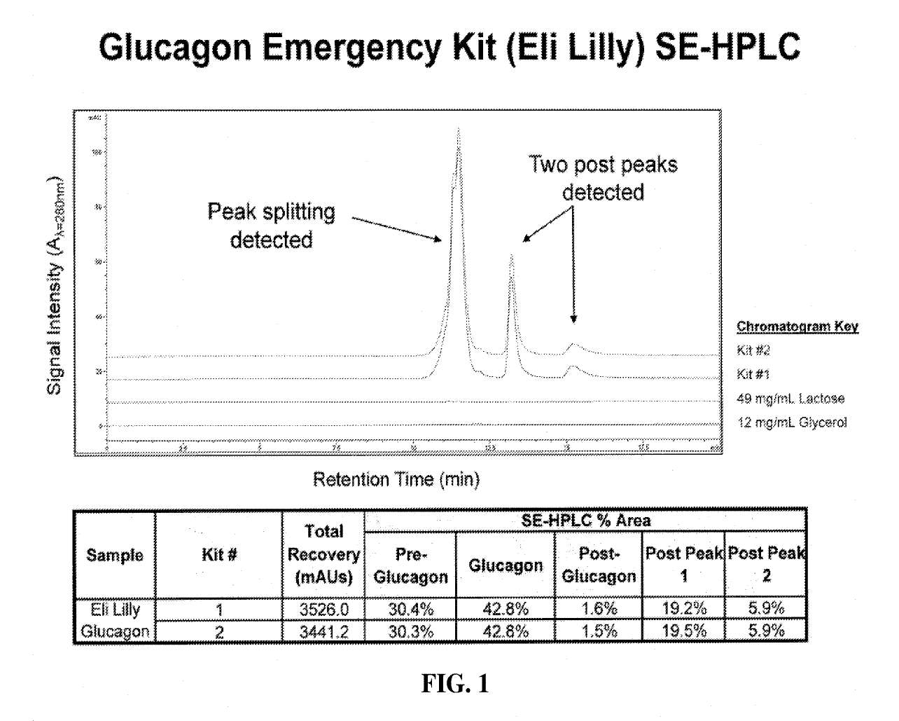

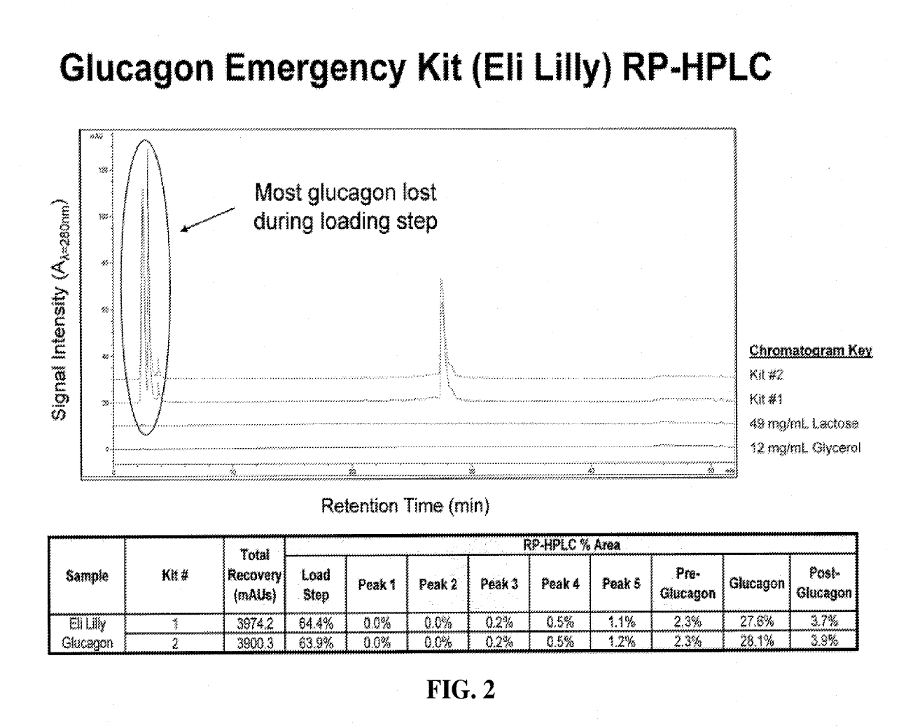

Two Glucagon Emergency Kits manufactured by Eli Lilly and Co. (each bearing Lot #A757B28F, Exp. 05/2012) containing 1 mg of glucagon, 49 mg of lactose, and HCl (lyophilized powder in vial) were reconstituted with the included diluent in the pre-filled syringe containing 12 mg/mL glycerin, HCl, and water for injection (Lot #A717859, Exp. 02/2013). High-performance liquid chromatography (HPLC) was performed on reconstituted samples according to standard methods, filling HPLC vial inserts with 90 μL of reconstituted sample and incubating for 4-6 hours at refrigerated temperatures, then injecting 10 μL of sample for analysis by size exclusion (SE)-HPLC and reverse phase (RP)-HPLC. As shown in FIG. 1, SE-HPLC analysis indicated that in less than 6 hours, the glucagon main peak purity dropped to only 42.8%. Two post-glucagon peaks were observed and main peak splitting was also observed. As shown in FIG. 2, RP-HPLC analysis indicated that in less than 6 hours, the glucagon main peak purity dropped to 27-28%. Most of the glucagon was lost during the loading step, likely due to fibrillation of the glucagon. This experiment demonstrates that the typical commercially available glucagon product is not stable for an extended period of time once reconstituted, and thus is not suitable for chronic dosing of glucagon.

### Ionization Stabilized Glucagon Composition

In this example, glucagon solutions were prepared by dissolving glycine hydrochloride (CAS No. 6000-43-7) directly in DMSO (CAS No. 67-68-5) at 5 mM, 10 mM, and 20 mM concentrations, followed by dissolution of glucagon powder (MW=3483 g/mol; Bachem AG) to a peptide concentration of 5 mg/mL. The prepared sample solutions are shown in Table 1:

TABLE 1
Glucagon sample solutions prepared by dissolving both
glycine hydrochloride and glucagon powder directly in DMSO.

| Glucagon Concentration | Solvent | Added Excipient |
|---|---|---|
| 5 mg/mL | DMSO | 5 mM Glycine Hydrochloride |
| 5 mg/mL | DMSO | 10 mM Glycine Hydrochloride |
| 5 mg/mL | DMSO | 20 mM Glycine Hydrochloride |

The reversed-phase high performance liquid chromatography (RP-HPLC) method used to assess chemical stability was a gradient method with mobile phases A and B respectively consisting of 0.1% (v/v) TFA (trifluoroacetic acid) in water and 0.1% (v/v) TFA in acetonitrile. A C8 column (BioBasic™-8; ThermoScientific) (4.6 mm I.D.×250 mm length, 5 micron particle size) was used with a column temperature of 37° C., a 1.0 mL/min flow rate, 6-μL sample injection volume and 280-nm detection wavelength.

Visual observation indicated that following six weeks (42 days) of storage at 40° C., the sample solutions containing glycine hydrochloride as a formulation excipient remained clear and colorless, and did not exhibit any precipitation and/or gelation. The stability of 5 mg/mL glucagon formulations were assessed via RP-HPLC as described above.

The sample formulations prepared with varying concentrations of glycine hydrochloride were sealed in 2-mL CZ vials (Crystal-Zenith, West Pharmaceuticals, PA, USA) with 13-mm FluroTec® stoppers (rubber stoppers coated with a fluorocarbon film, produced by West Pharmaceuticals) and stored at 40° C. for up to 6 weeks. The solutions were compared with 5 mg/mL glucagon formulations prepared via drying from a non-volatile buffer and reconstituting in DMSO (the pH memory formulations as described in Prestrelski '644), and direct dissolution of glucagon in DMSO (the method as described in Stevenson '547). The stability of the formulations are presented as glucagon purity and shown in Table 2 below.

TABLE 2
Stability (provided as peptide purity) of
5 mg/mL glucagon solutions stored at 40° C.

| | | | | | Direct |
|---|---|---|---|---|---|
| | Glycine HCl Concentration | | | pH Memory | Dissolution |
| Time Point | 5 mM | 10 mM | 20 mM | Formulation | In DMSO |
| Day 1 | 100% | 100% | 100% | 100% | Formed Gel |
| Day 14 | 99.7% | 99.5% | 99.3% | 99.4% | — |
| Day 42 | 97.8% | 97.0% | 97.0% | 96.8% | — |

Within 24 hours at room temperature, the 5 mg/mL glucagon solutions (approximately 0.45% w/w) prepared by direct dissolution of glucagon powder in DMSO exhibited physical aggregation, as noted by the formation of insoluble material (FIG. 1). By contrast, solutions prepared with 5 mg/mL glucagon powder dissolved in DMSO in the presence of 5.0 mM glycine HCl remained clear (i.e. free of precipitation) and colorless throughout the examined incubation period (6 weeks at 40° C.). Glucagon formulations that had previously been lyophilized from a buffered aqueous solution containing 1 mg/mL glucagon, 2 mM glycine and 1% (w/v) trehalose prior to reconstitution to 5-fold the initial concentration with DMSO (i.e., the composition in the aprotic polar solvent system following reconstitution was 5 mg/mL glucagon, 10 mM glycine, and 5% (w/v) trehalose) also exhibited a glucagon purity of approximately 97% following six weeks of storage at 40° C.

Accordingly, the compositions prepared by the method of the present invention provide enhanced stability compared to the prior art methods of direct dissolution of the peptide powder in an aprotic polar solvent. Further, the formulations of the present invention may provide an alternative pathway for preparing highly-concentrated, stable glucagon formulations in aprotic polar solvent systems without the need for drying the peptide from a buffered aqueous solution prior to dissolution in the aprotic polar solvent system.

Preparation of Dry Ionization Stabilized Glucagon Compositions at 5 mg/ml

In this example glucagon solutions were prepared at a concentration of 5 mg/mL by dissolving glucagon powder in DMSO that included different concentrations of added hydrochloric acid, ranging from 0.001 M (1 mM) to 0.01 M (10 mM). To minimize the amount of water added to the formulation, 5 N HCl was utilized to prepare 10 mM and 5.6 mM HCl in DMSO solutions, while 1 N HCl was used to prepare the 3.2 mM, 1.8 mM, and 1.0 mM solutions. As an example, the 10 mM HCl in DMSO solution was prepared by adding 20 μL of 5 N HCl to 9.98 mL of DMSO (neat), while the 1.0 mM HCl in DMSO solution was prepared by adding 10 μL of 1 N HCl to 9.99 mL of DMSO (neat). Samples of each formulation were stored in CZ vials and incubated at 40° C.

Following both 28 and 58 days of storage the chemical stability of the peptide was assessed by RP-HPLC and the purity reported in Table 3. The addition of 1.0 mM HCl was insufficient to prevent the formation of insoluble aggregates in the 5 mg/mL glucagon solutions, and accordingly the chemical stability of these samples were not measured. Conversely, the glucagon molecule exhibited relatively rapid chemical degradation when 10 mM HCl was added to the solution. Decreasing the added HCl concentration in the solution increased the overall stability of the glucagon molecule, with the 3.2 mM and 1.8 mM HCl solutions exhibiting the highest stability over the examined time period.

TABLE 3
Stability (provided as peptide purity) of
5 mg/mL Glucagon-DMSO Solutions Stored at 40° C.

| | Added | | |
|---|---|---|---|
| Glucagon | [HCl] | Day 28 | Day 58 |
| 5 mg/mL | 10.0 mM | 36.9% | 0% |
| 5 mg/mL | 5.6 mM | 90.8% | 85.3% |
| 5 mg/mL | 3.2 mM | 98.0% | 96.8% |
| 5 mg/mL | 1.8 mM | 98.3% | 97.4% |
| 5 mg/mL | 1.0 mM | Insoluble | Insoluble |
| | | Aggregates | Aggregates |

Preparation of Ionization Stabilized Glucagon Compositions at 5 mg/ml

Sample solutions were prepared by dissolving glucagon powder to a concentration of 5 mg/mL in DMSO which contained various added concentrations of glycine hydrochloride (CAS No. 6000-43-7), betaine hydrochloride (CAS No. 590-46-5), or hydrochloric acid (1 N; CAS No. 7647-01-0). The various concentrations of each ionization stabilizing excipient used to prepare the sample formulations are listed in Table 4. Samples of each formulation were stored in CZ vials and incubated at 40° C. Following 28 days of storage the chemical stability of the glucagon peptide was assessed by RP-HPLC and the purity reported in Table 4. This example demonstrates that the proton-donating ability of the added ionization stabilizing excipient (i.e. its ‘strength’) may influence the concentration required to stabilize the therapeutic molecule. Glucagon was selected as a model peptide due to its tendency to gel (i.e. form insoluble aggregates) when the molecule is insufficiently protonated. A concentration of up to 2 mM glycine hydrochloride was insufficient to prevent the formation of insoluble aggregates in the solution, though this concentration of both betaine hydrochloride and hydrochloric acid was sufficient to prevent the formation of insoluble aggregates following 28 days of storage at 40° C.

TABLE 4
Stability (provided as % peptide purity) of
5 mg/mL Glucagon-DMSO Solutions Stored at 40° C. for 28 days.

| | Ionization | | |
|---|---|---|---|
| Glucagon | Stabilizing | Added | |
| Powder | Excipient | Concentration | % Peptide Purity |
| 5 mg/mL | Glycine HCl | 0.5 mM | Insoluble |
| | | | Aggregates |
| 5 mg/mL | Glycine HCl | 1.0 mM | Insoluble |
| | | | Aggregates |
| 5 mg/mL | Glycine HCl | 2.0 mM | Insoluble |
| | | | Aggregates |
| 5 mg/mL | Glycine HCl | 3.0 mM | 98.5% |
| 5 mg/mL | Glycine HCl | 4.0 mM | 98.6% |
| 5 mg/mL | Glycine HCl | 5.0 mM | 99.1% |
| 5 mg/mL | Betaine HCl | 0.5 mM | Insoluble |
| | | | Aggregates |
| 5 mg/mL | Betaine HCl | 2.0 mM | 98.6% |
| 5 mg/mL | Betaine HCl | 5.0 mM | 98.4% |
| 5 mg/mL | HCl | 1.0 mM | Insoluble |
| | | | Aggregates |
| 5 mg/mL | HCl | 1.8 mM | 98.3% |
| 5 mg/mL | HCl | 3.2 mM | 98.0% |

Ionization Stabilized Glucagon Compositions at 5 mg/ml

The following example demonstrates the stability of a glucagon solution prepared according to the method of the present invention in the presence of added formulation components (e.g. inactive agents, excipients). Sample solutions were prepared by dissolving glucagon powder to a concentration of 5 mg/mL in DMSO which contained about 3.2 mM of added HCl (from a stock solution of 1 N HCl). To these solutions were added varying concentrations of moisture, as well as 5.5% (w/v) mannitol (CAS No. 69-65-8), and 1% (v/v) benzyl alcohol (CAS No. 100-51-6). The experimental samples examined are listed in Table 5.

Samples of each formulation were stored in CZ vials and incubated at room temperature (22-23° C.). Following 180 days (6 months) of storage the chemical stability of the glucagon peptide was assessed by RP-HPLC (according to the method described above) and the glucagon purity is reported in Table 5. This example demonstrates that additional formulation components (e.g. inactive agents, excipients) may be included in the formulation and still yield a stable formulation following approximately 6 months of storage at room temperature.

TABLE 5
Stability of 5 mg/mL Glucagon-DMSO Solutions
stored at room temperature for 180 days.
Stability is provided as glucagon purity as assessed by RP-HPLC

| | | | | Benzyl | |
|---|---|---|---|---|---|
| | Added | Moisture | Mannitol | Alcohol | % Glucagon |
| Glucagon | [HCl] | (% v/v) | (% w/v) | (% v/v) | Purity |
| 5 mg/mL | 3.2 mM | 0% | 0% | 0% | 98.2 |
| 5 mg/mL | 3.2 mM | 1% | 0% | 0% | 98.3 |
| 5 mg/mL | 3.2 mM | 3% | 0% | 0% | 98.1 |
| 5 mg/mL | 3.2 mM | 5% | 0% | 0% | 98.4 |
| 5 mg/mL | 3.2 mM | 1% | 5.5% | 0% | 98.6 |
| 5 mg/mL | 3.2 mM | 3% | 5.5% | 0% | 97.7 |
| 5 mg/mL | 3.2 mM | 5% | 5.5% | 0% | 98.9 |
| 5 mg/mL | 3.2 mM | 1% | 5.5% | 1% | 95.3 |
| 5 mg/mL | 3.2 mM | 3% | 5.5% | 1% | 96.9 |
| 5 mg/mL | 3.2 mM | 5% | 5.5% | 1% | 97.1 |

### Daily Glucagon Administration Reduces Weight Gain in Male Rats

For this study, glucagon formulation was prepared as follows:

Pre-lyophilized bulk drug product is prepared in an aqueous solution of 1.0 mg/mL glucagon, 2 mM glycine, and 1% w/w trehalose, at pH 3.0 as follows:
1. Combine glycine, trehalose, and water to prepare a 2.0 mM glycine, 1% (w/v) trehalose solution.
2. Measure pH and adjust to 3.0 with 1.0 N HCl if needed. If HCl added, check pH a second time.
3. Weigh glucagon powder (adjusted for the peptide content per glucagon CofA as peptide content of synthetic glucagon varies, but is approximately 94%) and dissolve glucagon powder in the buffer to a final concentration of 1.0 mg/mL glucagon. Verify concentration with UV-Vis_{A280} (AM500-54).
4. Filter solution with a Millipak 200 0.45 μm PVDF polishing filter to remove particulates.
5. Utilizing a bio-safety cabinet (BSC) fill 10.0 g aliquots into 20 mL glass vials, and stopper with single-vent lyo stoppers.
6. Place vials on a lyophilization tray, load into the BOC Edwards lyophilizer and initiate cycle.

The intermediate bulk drug product includes glucagon lyophiles reconstituted in DMSO that are pooled and aseptically filtered, creating a formulation of 5.0 mg/mL glucagon, 10 mM glycine, 5.0% w/w trehalose, and 94.5% w/w DMSO, with “pH memory” 3.0. All materials used in intermediate drug product formulation steps are DMSO compatible.
1. Obtain 500 mL DMSO to create the non-aqueous reconstitution solution.
2. Dissolve individual glucagon lyophiles by adding reconstitution solution to each vial, with a target concentration of 5.0 mg/mL glucagon (and a final trehalose concentration of 5.00%).
3. Pool reconstituted vials into a glass filling vessel and mix.
4. Assay for UV-Vis_{A280} (AM500-54) to determine if further dilution with the solvent mixture is required to achieve the target concentration.
5. If dilution with the solvent mixture is required, again assay for UV-Vis_{A280}.
6. Place batch in BSC and sterile filter using a Meissner 0.22 μm capsule filter with DMSO-compatible PTFE membrane.

TABLE 6

| Component | Concentration (% w/w) |
|---|---|
| Glucagon | 0.45 |
| Glycine | ≤0.1 |
| Trehalose | 5.00 |
| Dimethyl sulfoxide (dmso) | 94.54 |
| Hydrochloric acid¹ | |
| Total: | 100.00 |

¹variable depending on pH adjustment required: usually ~5 ml (≤0.1% w/w) per batch

Fifteen male Sprague Dawley rats were dosed daily with vehicle (a DMSO solution containing only glycine and trehalose) or 0.5, 1.0, or 2.0 mg/kg/day of Xeris' Glucagon for 91 days. The rats were at a starting age that would be considered pre-adult, but were well into the adult-stage by the middle of dosing period. They were maintained in standard rat cages and thus should be considered sedentary throughout their lives.

Multiple observations and clinical pathology endpoints were evaluated in this study. These endpoints included daily weights, which were formally recorded each week. A separate group of 9 rats each contributed 300 μl of whole blood. Individual rats contribute 3 blood samples. As a toxicokinetic curve requires 7-9 blood samples, 9 rats per dose-group were evaluated to complete an entire drug clearance curve.

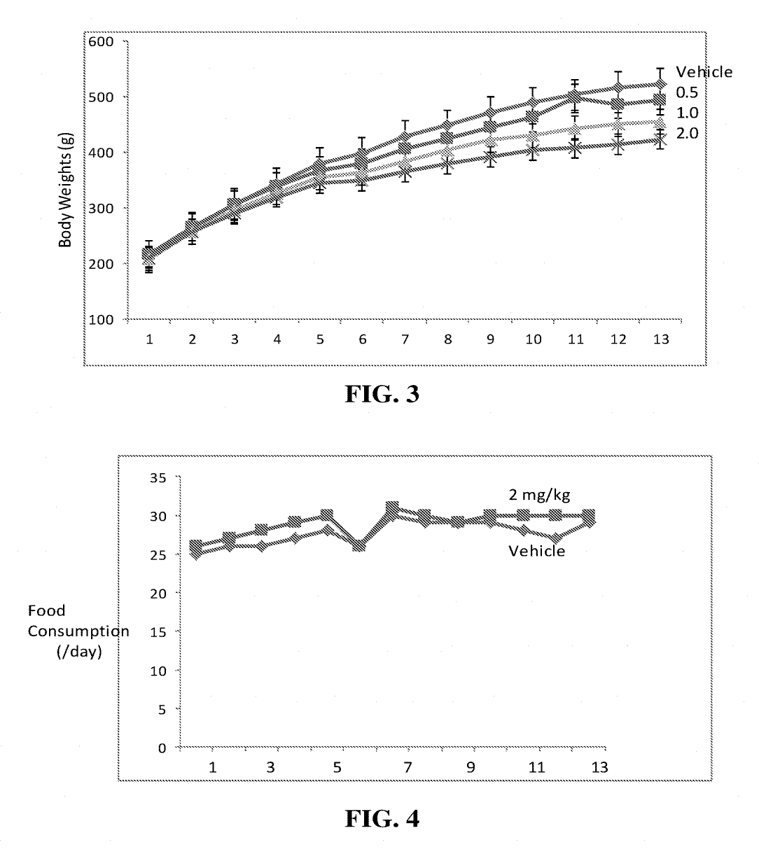

The weekly increase in weights for each group of rats is shown in FIG. 3. The data indicates a dose-related decrease in weight-gain where the last 7 points in the 2 mg/kg/day group were statistically lower relative to the vehicle. Statistical significance was evident at each dose at the end of the study. A few time points at the end of the study were also statistically different in the mid-dose group (see Table 7). The decreased weight gain noted in the glucagon-treated groups was not accompanied by any change in food consumption relative to the vehicle group (FIG. 4), indicating that in rats a mechanism other than food consumption, and specific to glucagon, was involved in the weight gain reduction.

Because of the amount of data present in FIG. 3, the points of significance are listed in Table 7 below. Statistical methods were analysis of variance with repeated measures. When F ratios indicated significance across doses, the Newman-Keuls test was used to determine which points were different from vehicle.

TABLE 7
Weeks at Which Weight gain Was Significantly Different from vehicle

| | Dose (mg/kg/day) | | |
|---|---|---|---|
| | 0.5 | 1.0 | 2.0 |
| Weeks where treated | 12 | 10, 11, 12, 13 | 6, 7, 8, 9, 10, 11, 12, |
| group was | | | 13 |
| significantly less than | | | |
| vehicle | | | |

P < 0.05 vs vehicle

Recovery from Glucagon Treatment Reduces the Reduction in Weight Gain Relative to Vehicle

Five rats per group were allowed to recover from treatment for 4 weeks prior to sacrifice. The body weights in each of the recovery groups are indicated in Table 8. The data indicate that although the rats that had previously received vehicle grew slightly during the recovery period, the rats that had been treated with glucagon grew faster to the point that the high dose group was no longer significantly different from the vehicle group.

TABLE 8
Body Weights During One Month Recovery Period
After Termination of Dosing

| | |
|---|---|
| Recovery | Dose (mg/kg) |

| Week | 0 | 0.5 | 1.0 | 2.0 |
|---|---|---|---|---|
| 14 | 526 | 511* | 496* | 446* |
| 15 | 534 | 522* | 503* | 489* |
| 16 | 545 | 526* | 513* | 529 |
| 17 | 556 | 532* | 522 | 541 |

P < 0.5 vs vehicle

Glucagon Administration does not Affect Food Consumption in Rats

Food consumption was measured on a weekly basis throughout the study. The data, shown in FIG. 4 indicate no difference between rats treated with glucagon at 2 mg/kg/day and vehicle. Statistical analysis indicated no difference between food consumption for any dose group vs vehicle. Based on this data the decrease in weight gain in rats cannot be attributed to difference in food consumption, as the data suggests that glucagon does not affect appetite in rats.

### Glucagon Treatment Reduces Mediators of Fat Production

The ten rats per treatment group were analyzed for multiple indicators of toxicological or pharmacological change at both the midpoint of the study (Week 6) and at the end of the study (week 13). Blood was removed, plasma obtained and measurements made with a clinical chemistry analyzer. The data indicates that glucagon treatment resulted in a significant reduction in cholesterol and triglycerides (Table 9). The data indicates that glucagon treatment may be associated with lipid mobilization, an effect that may explain the reduction is weight gain observed during the study. The data also indicate a recovery from this effect indicating that the effect observed was glucagon specific.

TABLE 9
Cholesterol and Triglycerides Resulting from
Treatment with Xeris's Glucagon^{a}

| | |
|---|---|
| Week of | |
| Dosing | |
| (Cholesterol/ | Dose (mg/kg) |

| Triglycerides | 0 | 0.5 | 1.0 | 2.0 |
|---|---|---|---|---|
| 7 | 104/56 | 97/51 | 87*/45 | 71**/41* |
| 13 | 108/58 | 92/50 | 81*/43* | 66**/39** |
| Recovery | | | | |
| 17 | 113/61 | 97/55 | 89/49 | 98/50* |

^{a}Only two blood samples were obtained during treatment and then at the end of recovery
*P < .05 vs Vehicle;
**P < .01

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications, patents and PCT publications are incorporated herein by reference for all purposes.

1. A method for controlling or reducing body weight in a subject in need thereof, the method comprising: administering to the subject a low dose of a stable glucagon formulation in response to a hunger cue in the subject, wherein the glucagon formulation is stable for at least one week at controlled room temperature; thereby controlling or reducing body weight in the subject.

2. The method of claim 1, wherein the subject is diabetic.

3. The method of claim 1, wherein the subject is normoglycemic.

4. The method of claim 1, wherein the subject is a human adult.

5. The method of claim 4, wherein the glucagon formulation is administered to the subject at a dose of about 50 μg to about 200 μg.

6. The method of claim 5, wherein the glucagon formulation is administered to the subject at a dose of about 150 μg.

7. The method of claim 1, wherein the subject is a human child.

8. The method of claim 7, wherein the glucagon formulation is administered to the subject at a dose of about 5 μg to about 150 μg.

9. The method of claim 1, wherein the glucagon formulation is stable for at least one month at controlled room temperature.

10. The method of claim 1, wherein the glucagon formulation is reconstituted with a pharmaceutically acceptable carrier

11. The method of claim 1, wherein the glucagon formulation comprises an ionization stabilizing excipient, wherein (i) the glucagon, glucagon analogue, or salt thereof is dissolved in the aprotic solvent in an amount from about 0.1 mg/mL up to the solubility limit of the glucagon, glucagon analogue, or salt thereof, and (ii) the ionization stabilizing excipient is dissolved in the aprotic solvent in an amount to stabilize the ionization of the glucagon peptide or salt thereof.

12. The method of claim 11, wherein the ionization stabilizing excipient is at a concentration of 0.1 mM to less than 100 mM.

13. The method of claim 11, wherein the ionization stabilizing excipient is a mineral acid.

14. The method of claim 13, wherein the mineral acid is hydrochloric acid.

15. The method of claim 11, wherein the aprotic solvent is DMSO.

16. The method of claim 11, wherein the aprotic solvent is a deoxygenated aprotic solvent.

17. The method of claim 11, wherein the ionization stabilizing excipient is HCl and the aprotic solvent is DMSO.

18. The method of claim 11, wherein the composition has a moisture content of less than 10, 5, or 3%.

19. The method of claim 11, wherein the composition further comprises a preservative at less than 10, 5, or 3% w/v.

20. The method of claim 19, wherein the preservative is benzyl alcohol.

21. The method of claim 11, wherein the composition further comprises a sugar alcohol at less than 10, 5, or 3% w/v.

22. The method of claim 21, wherein the sugar alcohol is mannitol.

23.-158. (canceled)
